SEQUENCE LISTING (104 sequences)

SEQ ID NO: 1 - DNA, 60 bp, Artificial Sequence
  artificial sequence (protospacer sequence)
atacaataaactagtaaaggcccaactgtggatatatctgaggcctgtcaagactcctgc

SEQ ID NO: 2 - DNA, 47 bp, Artificial Sequence
  artificial sequence (protospacer sequence)
aaactagtaaaggcccaactgtggatatatctgaggcctgtcaagac

SEQ ID NO: 3 - DNA, 46 bp, Artificial Sequence
  artificial sequence (protospacer sequence)
aaactagtaaaggcccaactgtggatatactgaggcctgtcaagac

SEQ ID NO: 4 - DNA, 45 bp, Artificial Sequence
  artificial sequence (protospacer sequence)
aaactagtaaaggcccaactgtggatatctgaggcctgtcaagac

SEQ ID NO: 5 - DNA, 44 bp, Artificial Sequence
  artificial sequence (protospacer sequence)
aaactagtaaaggcccaactgtggatatcgaggcctgtcaagac

SEQ ID NO: 6 - DNA, 37 bp, Artificial Sequence
  artificial sequence (protospacer sequence)
aaactagtaaaggcccaactctgaggcctgtcaagac

SEQ ID NO: 7 - DNA, 35 bp, Artificial Sequence
  artificial sequence (protospacer sequence)
aaactagtaaaggcccaactgaggcctgtcaagac

SEQ ID NO: 8 - DNA, 48 bp, Artificial Sequence
  artificial sequence (protospacer sequence)
aaactagtaaaggcccaactgtggatatattctgaggcctgtcaagac

SEQ ID NO: 9 - DNA, 23 bp, Artificial Sequence
  synthesized sequence
cccaactgtggatatatctgagg

SEQ ID NO: 10 - DNA, 120 bp, Artificial Sequence
  synthesized sequence
tttaaatttagctctaagatacaatacaataaactagtaaaggcccaactgtggatatatctgaggcctgtcaagactcctgcgacagtgtttgtgcaaatcctgagactcatcaaaccc

SEQ ID NO: 11 - DNA, 120 bp, Artificial Sequence
  synthesized sequence
tttaaatttagctctaagatacaatacaataaactagtaaaggcccaaatgtggatatatctgaggcctgtcaagactcctgcgacagtgtttgtgcaaatcctgagactcatcaaaccc

SEQ ID NO: 12 - DNA, 120 bp, Artificial Sequence
  synthesized sequence
tttaaatttagcgctaagatacaatacaataaactagtaaaggcccaactgtggatatatctgaggcctgtcaagactcctgcgacagtgtttgtgcaaatcctgagactcatcaaaccc

SEQ ID NO: 13 - DNA, 120 bp, Artificial Sequence
  synthesized sequence
tttaaatttagctctaagatacaatacaataaactagtgaaggcccaactgtggatatatctgaggcctgtcaagactcctgcgacagtgtttgtgcaaatcctgagactcatcaaaccc

SEQ ID NO: 14 - DNA, 120 bp, Artificial Sequence
  synthesized sequence
tttaaatttagctctaagatacaatacaataaactagtaaaggcccaactgtggatatatctgaggcctgtcaagactcctgcgacagtgtttgttcaaatcctgagactcatcaaaccc

SEQ ID NO: 15 - DNA, 120 bp, Artificial Sequence; identical to SEQ ID NO: 10
  synthesized sequence

SEQ ID NO: 16 - DNA, 108 bp, Artificial Sequence
  synthesized sequence
tttaaatttagctctaagatacaatacaataaactagtaaaggcccaactgaggcctgtcaagactcctgcgacagtgtttgtgcaaatcctgagactcatcaaaccc

SEQ ID NO: 17 - DNA, 120 bp, Artificial Sequence; identical to SEQ ID NO: 12
  synthesized sequence

SEQ ID NO: 18 - DNA, 120 bp, Artificial Sequence; identical to SEQ ID NO: 11
  synthesized sequence

SEQ ID NO: 19 - DNA, 120 bp, Artificial Sequence
  synthesized sequence
tttaaatttagctctaagatacaatacaaaaaactagtaaaggcccaactgtggatatatctgaggcctgtcaagactcctgcgacagtgtttgtgcaaatcctgagactcatcaaaccc

SEQ ID NO: 20 - DNA, 120 bp, Artificial Sequence; identical to SEQ ID NO: 10
  synthesized sequence

SEQ ID NO: 21 - DNA, 108 bp, Artificial Sequence; identical to SEQ ID NO: 16
  synthesized sequence

SEQ ID NO: 22 - DNA, 120 bp, Artificial Sequence
  synthesized sequence
tttaaatttagctctaagatacaatacaataaattagtaaaggcccaactgtggatatatctgaggcctgtcaagactcctgcgacagtgtttgtgcaaatcctgagactcatcaaaccc

SEQ ID NO: 23 - DNA, 120 bp, Artificial Sequence; identical to SEQ ID NO: 13
  synthesized sequence

SEQ ID NO: 24 - DNA, 120 bp, Artificial Sequence
  synthesized sequence
tttaaatttagctctaagatacaatacaataaactagtaaaggcccaactgtggatatatctgaggcctgtcaagactcctgcgacagtgtttgtgcaaatcctgagactcatcaagccc

SEQ ID NO: 25 - DNA, 120 bp, Artificial Sequence; identical to SEQ ID NO: 10
  synthesized sequence

SEQ ID NO: 26 - DNA, 108 bp, Artificial Sequence; identical to SEQ ID NO: 16
  synthesized sequence

SEQ ID NO: 27 - DNA, 108 bp, Artificial Sequence
  synthesized sequence
tttaaatttagctctaagatacaatacaataaactagtaaaggcccaactgtggcctgtcaagactcctgcgacagtgtttgtgcaaatcctgagactcatcaaaccc

SEQ ID NO: 28 - DNA, 12 bp, Artificial Sequence
  target sequence
atatatccacag

SEQ ID NO: 29 - DNA, 12 bp, Artificial Sequence
  target sequence
ctgtggatatat

SEQ ID NO: 30 - protein, 371 aa, Artificial Sequence
  pro-myostatin protein
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 31 - protein, 371 aa, Artificial Sequence
  pro-myostatin protein
MQKLQIYVYIYLFMLIVAGPVDLNENSEQKENVEKEGLCNACMWRQNTKSSRLEAIKIQILSKLRLETAPNISKDAIRQLLPKAPPLRELIDQYDVQRDDSSDGSLEDDDYHATTETIITMPTESDLLMQVEGKPKCCFFKFSSKIQYNKVVKAQLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 32 - protein, 372 aa, Artificial Sequence
  pro-myostatin protein
MMQKLQMYVYIYLFMLIAAGPVDLNEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKGYKANYCSGECEFVFLQKYPHTHLVHQANPRGSAGPCCTPTKMSPINMLYFNGKEQIIYGKIPAMVVDRCGCS

SEQ ID NO: 33 - protein, 371 aa, Artificial Sequence
  pro-myostatin protein
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 34 - protein, 108 aa, Artificial Sequence
  mature-myostatin protein
FGLDCDEHSTESRCCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 35 - protein, 95 aa, Artificial Sequence
  mature-myostatin protein
CCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 36 - protein, 95 aa, Artificial Sequence
  mature-myostatin protein
CCRYPLTVDFEAFGWDWIIAPKGYKANYCSGECEFVFLQKYPHTHLVHQANPRGSAGPCCTPTKMSPINMLYFNGKEQIIYGKIPAMVVDRCGCS

SEQ ID NO: 37 - protein, 95 aa, Artificial Sequence; identical to SEQ ID NO: 35
  mature-myostatin protein

SEQ ID NO: 38 - DNA, 20 bp, Artificial Sequence
  target sequence
cggagtctatataggtgtca

SEQ ID NO: 39 - DNA, 20 bp, Artificial Sequence
  target sequence
ggagtctatataggtgtcaa

SEQ ID NO: 40 - DNA, 20 bp, Artificial Sequence
  target sequence
gggttgacacctatatagac

SEQ ID NO: 41 - DNA, 20 bp, Artificial Sequence
  target sequence
ccggagtctatataggtgtc

SEQ ID NO: 42 - DNA, 20 bp, Artificial Sequence
  target sequence
tccgggttgacacctatata

SEQ ID NO: 43 - DNA, 20 bp, Artificial Sequence
  target sequence
ctatataggtgtcaacccgg

SEQ ID NO: 44 - DNA, 20 bp, Artificial Sequence
  target sequence
gtgtcaacccggaaatgatc

SEQ ID NO: 45 - DNA, 20 bp, Artificial Sequence
  target sequence
cagagtctatataggtgtca

SEQ ID NO: 46 - DNA, 20 bp, Artificial Sequence
  target sequence
agagtctatataggtgtcaa

SEQ ID NO: 47 - DNA, 20 bp, Artificial Sequence
  target sequence
ccagagtctatataggtgtc

SEQ ID NO: 48 - DNA, 20 bp, Artificial Sequence
  target sequence
gtgtcaacccggaaatgatg

SEQ ID NO: 49 - DNA, 20 bp, Artificial Sequence
  target sequence
cagagtttatataggtatca

SEQ ID NO: 50 - DNA, 20 bp, Artificial Sequence
  target sequence
agagtttatataggtatcaa

SEQ ID NO: 51 - DNA, 20 bp, Artificial Sequence
  target sequence
tacctatataaactctgggc

SEQ ID NO: 52 - DNA, 20 bp, Artificial Sequence
  target sequence
tccgggttgatacctatata

SEQ ID NO: 53 - DNA, 20 bp, Artificial Sequence
  target sequence
ccagagtttatataggtatc

SEQ ID NO: 54 - DNA, 20 bp, Artificial Sequence
  target sequence
ttatataggtatcaacccgg

SEQ ID NO: 55 - DNA, 20 bp, Artificial Sequence
  target sequence
gtatcaacccggaaatgatg

SEQ ID NO: 56 - DNA, 20 bp, Artificial Sequence
  target sequence
cagactctatataggtgtca

SEQ ID NO: 57 - DNA, 20 bp, Artificial Sequence
  target sequence
agactctatataggtgtcaa

SEQ ID NO: 58 - DNA, 20 bp, Artificial Sequence
  target sequence
ccagactctatataggtgtc

SEQ ID NO: 59 - DNA, 20 bp, Artificial Sequence
  target sequence
tctatataggtgtcaacccg

SEQ ID NO: 60 - DNA, 20 bp, Artificial Sequence
  target sequence
gtgacaacccgaaaatgatg

SEQ ID NO: 61 - RNA, 77 nt, Artificial Sequence
  Streptococcus pyogenes guide RNA
guuuuagucccugaaaagggacuaaaauaaagaguuugcgggacucugcgggguuacaauccccuaaaaccgcuuuu

SEQ ID NO: 62 - RNA, 20 nt, Artificial Sequence
  guide sequence
gccucagauauauccacagu

SEQ ID NO: 63 - RNA, 20 nt, Artificial Sequence
  guide sequence
ccucagauauauccacaguu

SEQ ID NO: 64 - RNA, 20 nt, Artificial Sequence
  guide sequence
cccaacuguggauauaucug

SEQ ID NO: 65 - RNA, 20 nt, Artificial Sequence
  guide sequence
ggccucagauauauccacag

SEQ ID NO: 66 - RNA, 20 nt, Artificial Sequence
  guide sequence
aggcccaacuguggauauau

SEQ ID NO: 67 - RNA, 20 nt, Artificial Sequence
  guide sequence
gauauauccacaguugggcc

SEQ ID NO: 68 - RNA, 20 nt, Artificial Sequence
  guide sequence
cacaguugggccuuuacuag

SEQ ID NO: 69 - RNA, 20 nt, Artificial Sequence
  guide sequence
gucucagauauauccacagu

SEQ ID NO: 70 - RNA, 20 nt, Artificial Sequence
  guide sequence
ucucagauauauccacaguu

SEQ ID NO: 71 - RNA, 20 nt, Artificial Sequence
  guide sequence
ggucucagauauauccacag

SEQ ID NO: 72 - RNA, 20 nt, Artificial Sequence
  guide sequence
cacaguugggccuuuacuac

SEQ ID NO: 73 - RNA, 20 nt, Artificial Sequence
  guide sequence
gucucaaauauauccauagu

SEQ ID NO: 74 - RNA, 20 nt, Artificial Sequence
  guide sequence
ucucaaauauauccauaguu

SEQ ID NO: 75 - RNA, 20 nt, Artificial Sequence
  guide sequence
auggauauauuugagacccg

SEQ ID NO: 76 - RNA, 20 nt, Artificial Sequence
  guide sequence
aggcccaacuauggauauau

SEQ ID NO: 77 - RNA, 20 nt, Artificial Sequence
  guide sequence
ggucucaaauauauccauag

SEQ ID NO: 78 - RNA, 20 nt, Artificial Sequence
  guide sequence
aauauauccauaguugggcc

SEQ ID NO: 79 - RNA, 20 nt, Artificial Sequence
  guide sequence
cauaguugggccuuuacuac

SEQ ID NO: 80 - RNA, 20 nt, Artificial Sequence
  guide sequence
gucugagauauauccacagu

SEQ ID NO: 81 - RNA, 20 nt, Artificial Sequence
  guide sequence
ucugagauauauccacaguu

SEQ ID NO: 82 - RNA, 20 nt, Artificial Sequence
  guide sequence
ggucugagauauauccacag

SEQ ID NO: 83 - RNA, 20 nt, Artificial Sequence
  guide sequence
agauauauccacaguugggc

SEQ ID NO: 84 - RNA, 20 nt, Artificial Sequence
  guide sequence
cacaguugggcuuuuacuac

SEQ ID NO: 85 - DNA, 20 bp, Artificial Sequence
  MSTN F primer
aacagcgagcagaaggaaaa

SEQ ID NO: 86 - DNA, 20 bp, Artificial Sequence
  MSTN R primer
ccaggcgaagtttactgagg

SEQ ID NO: 87 - DNA, 20 bp, Artificial Sequence
  GAPDH F primer
ggcgtgaaccacgagaagta

SEQ ID NO: 88 - DNA, 54 bp, Artificial Sequence
  GAPDH R primer
gtgactggagttcagacgtgtgctcttccgatcttgctctgccaaataccagtg

SEQ ID NO: 89 - DNA, 20 bp, Artificial Sequence
  MSTN F primer
gaggtgttcgttcgtttttc

SEQ ID NO: 90 - DNA, 20 bp, Artificial Sequence
  MSTN R primer
ctaccagtttcctgtgctta

SEQ ID NO: 91 - DNA, 20 bp, Artificial Sequence
  off target F primer
tcagcacagaaaaggtgagg

SEQ ID NO: 92 - DNA, 20 bp, Artificial Sequence
  off target R primer
gagacggacacaactgagca

SEQ ID NO: 93 - DNA, 20 bp, Artificial Sequence
  off target F primer
tgagcccctactttgtggac

SEQ ID NO: 94 - DNA, 20 bp, Artificial Sequence
  off target R primer
gttttctggtaaggggtgca

SEQ ID NO: 95 - DNA, 22 bp, Artificial Sequence
  off target F primer
ttgaaaacctagtggggaaaaa

SEQ ID NO: 96 - DNA, 20 bp, Artificial Sequence
  off target R primer
gcactctcaaacactgtggc

SEQ ID NO: 97 - DNA, 20 bp, Artificial Sequence
  off target F primer
tccttgcaccttccaaaatc

SEQ ID NO: 98 - DNA, 20 bp, Artificial Sequence
  off target R primer
atctgcgtgtaactccagcc

SEQ ID NO: 99 - DNA, 20 bp, Artificial Sequence
  off target F primer
tcacccattccagtccattt

SEQ ID NO: 100 - DNA, 20 bp, Artificial Sequence
  off target R primer
cctctaatgccctcttgcag

SEQ ID NO: 101 - DNA, 20 bp, Artificial Sequence
  MSTN-1 F primer
gaggtgttcgttcgtttttc

SEQ ID NO: 102 - DNA, 20 bp, Artificial Sequence
  MSTN-1 R primer
taagcacaggaaactggtag

SEQ ID NO: 103 - DNA, 53 bp, Artificial Sequence
  MSTN-2 F primer
acactctttccctacacgacgctcttccgatctaacgcaagtggaaggaaaac

SEQ ID NO: 104 - DNA, 54 bp, Artificial Sequence; identical to SEQ ID NO: 88
  MSTN-2 R primer

(19) United States
(12) Patent Application Publication
(10) Pub. No.: US 2025/0280804 A1
(43) Pub. Date: Sep. 11, 2025

(54) TRANSGENIC ANIMAL HAVING MODIFIED MYOSTATIN GENE

(71) Applicants: LART BIO CO., LTD, Seoul (KR); SEOUL NATIONAL UNIVERSITY, Seoul (KR); KYUNGSANGBUKDO, Yeongiu-si Gyeongsangbuk-do (KR)

(72) Inventors: Goo JANG, Seoul (KR); Gyeong Min Kim, Seoul (KR); Dong Hyeok Kwon, Seoul (KR); Ji Hyun Lee, Seoul (KR); Won You Lee, Hanam-si, Gyeonggi-do (KR); Ji Hyun Park, Hanam-si, Gyeonggi-do (KR); Soo Young Yum, Seoul (KR); Joon Ho Moon, Seoul (KR); Jun Koo Yi, Daegu (KR); Dae Jin Jung, Andong-si, Gyeongsangbuk-do (KR); Jae Jung Ha, Seoul (KR); Dae Hyun Kim, Yecheon-gun (KR)

(21) Appl. No.: 18/265,086

(22) PCT Filed: Dec. 3, 2021
(86) PCT No.: PCT/KR21/18176

§ 371 Date: Jun. 2, 2023

(30) Foreign Application Priority Data
Dec. 3, 2020 (KR) 10-2020-0167296

(51) Int. Cl. A01K 67/0276; C07K 14/475; C12N 5/071; C12N 9/22; C12N 15/11; C12N 15/85

(52) U.S. Cl. CPC A01K 67/0276; C07K 14/475; C12N 5/0602; C12N 9/226; C12N 15/111; C12N 15/8509; A01K 2217/054; A01K 2227/101; C12N 2310/20; C12N 2510/00

(57) ABSTRACT

The present application relates to an animal or cell having a myostatin gene in which 12 base pairs of the second exon are deleted. The present application may also comprise a composition capable of manipulating the deletion of 12 base pairs of a myostatin gene to construct the animal or the cell. The present application also relates to use of the composition for increasing muscle.

### CROSS-REFERENCE TO RELATED APPLICATIONS

This application is a national-stage filing under 35 U.S.C. § 371 of PCT Application No. PCT/KR2021/018176 filed on Dec. 3, 2021, which claims the benefit of priority to Korean Application No. 10-2020-0167296 filed on Dec. 3, 2020. The entire contents of PCT/KR2021/018176 are incorporated herein by reference in their entirety.

### INCORPORATION BY REFERENCE OF MATERIAL SUBMITTED ELECTRONICALLY

Incorporated by reference in its entirety is a computer-readable nucleotide/amino acid sequence listing submitted electronically in ASCII format. Said ASCII copy, created on Jan. 23, 2024, is named 18265086_1_1.txt, and is 35,447 bytes in size.

### TECHNICAL FIELD

This application relates to a transgenic animal or cell having an artificially modified gene. The transgenic animal or cell has a myostatin gene with a deletion of 12 base pairs in the second exon.

This application relates to technologies related to the production of transgenic animals or cells.

### BACKGROUND ART

The ‘Belgian Blue’ is famous as a superior breed of cattle with well-developed muscles. The breed was created by accident through crossbreeding by Belgian breeders in the 19th century. Although they do not consume much feed compared to the wild type, they are characterized by a high proliferation of muscle cells for genetic reasons, resulting in a diet low in fat and high in protein. It has been reported that these characteristics are caused by modification of the myostatin gene (McPherron A C, Lawler A M, Lee S J (1997) Nature 387:83-90).

Myostatin implies a meaning of “muscle (myo)+inhibitor (statin)” from the name, and it is already known that myostatin protein inhibits muscle differentiation and growth as research continues. Myostatin has been studied to control the differentiation and growth of muscle cells through gene regulation using gene editing tools in various animal models.

Furthermore, in the case of the myostatin transgenic large animal, a high-quality of meat can be obtained, thereby increasing the utilization. However, deletion of the myostatin gene still has technical limitations with several side effects such as an enlarged heart, increased blood pressure, and short life span.

As a result of the above technical limitations, myostatin transgenic animals are not yet available for industrial use.

Accordingly, the present applicants have tried to obtain myostatin transgenic animals that are healthy and have increased muscle mass. As a result, the present disclosure was completed by confirming that the transgenic bovine of the present application have a specific variant in which myostatin is modified, and to be a transgenic animal without conventional side effects.

### RELATED ART LITERATURES

### Patent Literatures

(Patent Literature 1) CN 104531705A
(Patent Literature 2) CN 107034221A

### Non-Patent Literature

(Non-Patent Literature 1) Am J Physiol Endocrinol Metab. 2017 Mar. 1; 312 (3): E150-E160, Myostatin propeptide mutation of the hyper muscular compact mice decreases the formation of myostatin and improves insulin sensitivity.

### DISCLOSURE

### Technical Problem

One objective of the present application is to provide an animal having an artificially modified myostatin gene in which 12 base pairs of the second exon are deleted.

Another objective of the present application is to provide an embryo having an artificially modified myostatin gene in which 12 base pairs of the second exon are deleted.

Still another objective of the present application is to provide a composition for deleting 12 base pairs of the second exon of the myostatin gene.

Still another objective of the present application is to provide a use for inducing muscle growth in the muscles of animals using the composition.

### Technical Solution

In order to solve the above problems, the present specification provides a transgenic animal having a myostatin gene with a specific part artificially modified.

The modification may occur in the second exon of the myostatin gene.

The above modification is a deletion of 12 base pairs in the second exon, corresponding to the region encoding the amino acid sequence of leucine, tryptophan, isoleucine, and tyrosine, compared to the myostatin gene sequence in wild-type animals.

The nucleic acid encoding an amino acid sequence of the order leucine, tryptophan, isoleucine, and tyrosine may have one or more sequences encoding each amino acid.

That is, the sequence encoding leucine may be one selected from 5′-CTT-3′, 5′-CTC-3′, 5′-CTA-3′, or 5′-CTG-3′, and the sequence encoding tryptophan may be one selected from 5′-IGG-3′, the sequence encoding isoleucine may be one selected from 5′-ATT-3′, 5′-ATC-3′, or 5′-ATA-3′, and the sequence encoding tyrosine may be one selected from 5′-TAT-3′ or 5′-TAC-3′.

The amount of myostatin mRNA expression in the transgenic animal is lower than that in the wild-type animal.

In addition, the transgenic animal may express a mature myostatin protein having the same amino acid sequence as the wild-type animal.

The transgenic animal may have an increased amount of muscle outwardly compared to the wild-type animal.

The transgenic animal includes mammals.

The mammals include ungulates.

The ungulates include artiodactyls. The artiodactyls may include pigs, deer, cattle, sheep, and goats but are not limited thereto.

The mammals may include rodents. The rodents may include mice and rats but are not limited thereto.

Preferably, the transgenic animal in the present application is a bovine. The bovine expresses a pro-myostatin protein including the amino acid sequence represented by SEQ ID NO: 30.

In addition, the present application provides an engineered cell having an artificially modified myostatin gene with a specific part modified.

The transformation of the engineered cell may occur in the second exon of the myostatin gene.

The above modification is a deletion of 12 base pairs in the second exon, corresponding to the region encoding the amino acid sequence of leucine, tryptophan, isoleucine, and tyrosine, compared to the myostatin gene sequence in wild-type cells.

The nucleic acid encoding an amino acid sequence of the order leucine, tryptophan, isoleucine, and tyrosine may have one or more sequences encoding each amino acid.

That is, the sequence encoding leucine may be one of 5′-CTT-3′, 5′-CTC-3′, 5′-CTA-3′, or 5′-CTG-3′, and the sequence encoding tryptophan may be one of 5′-TGG-3′, the sequence encoding isoleucine may be one of 5′-ATT-3′, 5′-ATC-3′, or 5′-ATA-3′, and the sequence encoding tyrosine may be one of 5′-TAT-3′ or 5′-TAC-3′.

The engineered cells expressed less myostatin mRNA than wild-type cells.

In addition, the engineered cell may express a mature myostatin protein having the same amino acid sequence as the wild-type animal.

The cells may be embryonic cells, somatic cells, or stem cells.

The cells include but are not limited to, for example, oocytes, epithelial cells, fibroblasts, nerve cells, keratinocytes, hematopoietic cells, melanocytes, chondrocytes, macrophages, monocytes, muscle cells, and B lymphocytes, T lymphocytes, embryonic stem cells, embryonic germ cells, fetal-derived cells, placental cells, and embryonic cells. In addition, adult stem cells derived from various tissues of origin can be used, for example, tissue-derived stem cells from fat, uterus, bone marrow, muscle, placenta, cord blood, or skin (epithelium). Non-human host embryos can generally be embryos containing a 2-cell stage, a 4-cell stage, an 8-cell stage, a 16-cell stage, a 32-cell stage, a 64-cell stage, an aborted embryo, or a blastocyst.

The cells can be obtained from mammals.

Preferably, the cells of the present application can be obtained from a bovine.

The engineered cell may express the pro-myostatin protein having the amino acid sequence represented by SEQ ID NO: 30.

The present application provides a composition for modifying the myostatin gene.

To modify the myostatin gene, the composition may include
a guide RNA including a guide sequence that forms a complementary bond with a target sequence or a DNA encoding the guide RNA;
and a Cas protein or a nucleic acid sequence encoding the guide RNA.

The target sequence may include one or more selected from SEQ ID NO: 38 to SEQ ID NO: 60.

The guide sequence may include one or more selected from SEQ ID NO: 624 to SEQ ID NO: 84.

The Cas protein may be one selected from the group consisting of a Streptococcus pyogenes-derived Cas9 protein, a Staphylococcus aureus-derived Cas9 protein, or a Cas 12a protein (conventionally known as CPF1: Prevotella and Francisella 1) protein. The nucleic acid encoding the Cas protein may be any one selected from the group consisting of a Streptococcus pyogenes-derived Cas9 protein, a Staphylococcus aureus-derived Cas9 protein, or a nucleic acid encoding a Cas 12a protein (conventional CPF1: Prevotella and Francisella 1) protein.

The composition may be present in a plasmid vector in the form of DNA encoding a guide RNA and a Cas protein.

The composition may be present in a viral vector in the form of DNA encoding a guide RNA and a Cas protein.

At this time, the viral vector may be one or more selected from the group consisting of a retrovirus vector, a lentivirus vector, an adenovirus vector, an adeno-associated virus (AAV) vector, a vaccinia virus vector, a poxvirus vector, and a herpes simplex virus vector.

The gene manipulation composition may be in the form of a complex (RNP: ribonucleoprotein) including a guide RNA and a Cas protein.

In addition, the present application provides a method for preparing a cell or embryo having an artificially modified myostatin gene modified with the above composition.

The method for producing myostatin-engineered cells or embryos of the present application,

may include contacting the cell or embryo with the composition. The contacting step may be performed in vivo or ex vivo.

The contacting step may be performed by one or more methods selected from microinjection, electroporation, liposomes, plasmids, viral vectors, nanoparticles, and protein translation domain (PTD) fusion protein methods.

In addition, the present application provides a method for preparing an animal having an artificially modified myostatin gene.

A method of producing a myostatin transgenic animal of the present disclosure may include the steps of producing a transgenic embryo having the artificially modified gene by contacting the embryo with a composition as described above and transferring the transgenic embryo to a surrogate mother.

Animals produced by the above production method express less myostatin mRNA than wild-type animals.

The animal may be a mammal other than a human.

### Advantageous Effects

The myostatin transgenic animal of the present application can increase the amount of muscle compared to wild-type animals due to the low expression level of myostatin mRNA.

As a result, it is possible to provide high-quality meat with a low-fat content and a high protein content.

Conventional myostatin-mutated transgenic animals have various side effects such as a short life span, but the present application can provide a healthy myostatin-mutated transgenic animals without such various side effects.

In addition, the composition provided by the present application is a composition capable of modifying the myostatin gene, and may be capable of increasing muscle mass when injected into the tissue of an animal.

### DESCRIPTION OF DRAWINGS

FIG. 1 shows a diagram of the location of the modification in the myostatin gene and lists the protospacer sequences used in one example of the present disclosure.

FIG. 2 is a schematic diagram of a method for producing a transgenic embryo having an artificially modified myostatin gene in which 12 base pairs of the second exon are deleted.

FIG. 3 shows the confirmation of myostatin modification in an engineered embryo having a myostatin gene in which 12 base pairs are deleted in the second exon by T7E1 assay.

FIG. 4 shows the protospacer sequence of the myostatin gene and Sanger sequencing of myostatin-engineered embryos with a guide RNA containing a sequence that binds to its complementary target sequence, revealing multiple variants.

FIG. 5 shows different amounts of guide RNA or mRNA of Cas9 used to drive the deletion of 12 base pairs of the second exon of the myostatin gene to determine the most appropriate guide RNA and CAS9 mRNA amounts to proceed with the present application.

FIG. 6 shows a cattle with a myostatin gene deletion of 12 base pairs in the second exon, photographed for appearance checks once a month for one to four months after birth.

FIG. 7 shows the confirmation of myostatin modification in a cattle having a myostatin gene in which 12 base pairs are deleted in the second exon by T7E1 assay.

FIG. 8 shows five sequences for potential off-target sites identified by T7E1 assay in order to confirm an off-target effect that can be generated by CRISPR/Cas9. It was confirmed that neither hetero-knockout nor homo-knockout occurred for all five off-target positions by mixing in wild-type DNA or not mixing in wild-type DNA.

FIG. 9 shows deep sequencing results of 17 cattle born after implantation of an embryo generated by the method of FIG. 2 into the uterus of a surrogate mother, confirming a 12 base pair deletion in the myostatin gene.

FIG. 10 lists the deep sequencing results of a wild-type cattle as a negative control for a cattle with a myostatin gene that has a deletion of 12 base pairs in the second exon.

FIG. 11 lists the deep sequencing results for cattle No. 6, which has a myostatin gene with a deletion of 12 base pairs in the second exon.

FIG. 12 lists the deep sequencing results for cattle No. 14, which has a myostatin gene with a deletion of 12 base pairs in the second exon.

FIG. 13 lists the deep sequencing results for cattle No. 17, which has a myostatin gene with a deletion of 12 base pairs in the second exon.

FIG. 14 shows the amount of myostatin mRNA expression in cattles 14 and 17, which have a myostatin gene with a deletion of 12 base pairs in the second exon.

FIG. 15 shows a representative photograph of the validation of germline transfer of MSTN mutant females, production of MSTN mutant blastocysts derived from MSTN mutant cattle oocytes, and diagnosis of pregnancy by ultrasound machine at day 30.

FIG. 16 is a somatic cell image derived from follicular fluid obtained during OPU.

FIG. 17 shows T7E1 assay results and sequencing data from an MSTN mutant female blastocyst.

FIG. 18 shows T7E1 assay results and sequencing data from somatic cells in follicular fluid.

FIG. 19 shows a summary of semen from the MSTN male founder by Computer Assisted Semen Analysis.

FIG. 20 shows a photograph of a representative blastocyst as a validated result of germline transfer from an MSTN mutant male cattle.

FIG. 21 shows the mutation rate of the MSTN gene in blastocysts derived from in vitro fertilized MSTN mutant bull semen.

### BEST MODE

### Mode for Invention

In order to describe the content disclosed herein, several terms will be defined herein. In addition to these terms, other terms are defined elsewhere in this specification where necessary. Unless expressly defined otherwise herein, trade terms used herein shall have their art recognized meanings. In case of conflict, the definitions herein shall govern.

### Definition of Common Terms

### Conserved Region of the Myostatin Gene

The conserved region of the myostatin gene refers to a nucleic acid sequence encoding a common preserved region without modification) in the amino acid sequences of myostatin, across species during evolution.

In the present application, term “a conserved region of the myostatin gene by species” includes nucleic acid sequences encoding amino acids in the order of leucine, tryptophan, isoleucine, and tyrosine in conserved region of the myostatin (see Table 3).

The conserved region of the myostatin by species may have the same amino acid sequence, but there may be several base codons for the amino acids sequence according to the species. That is, a nucleic acid sequence encoding leucine may be one of 5′-CTT-3′, 5′-CTC-3′, 5′-CTA-3′, or 5′-CTG-3′, and a nucleic acid sequence encoding tryptophan may be one of 5′-TGG-3′, a nucleic acid sequence encoding isoleucine may be one of 5′-ATT-3′, 5′-ATC-3′, or 5′-ATA-3′, and a nucleic acid sequence encoding tyrosine may be one of 5′-TAT-3′ or 5′-TAC-3′.

Therefore, the nucleic acid sequence of the “conserved region of the myostatin gene by species” of the present application may be different for each species. In the present specification, the “conserved region of the myostatin gene” is sometimes abbreviated as “conserved region”.

### Transgenic Animal

The term the “transgenic animal” in the present application means an animal having an artificially modified myostatin gene.

In this application, “transgenic animal” has an artificially modified myostatin gene in which 12 base pairs of the second exon are deleted and expresses a mature myostatin protein with the same sequence as a wild-type animal.

The trait of the artificially modified myostatin gene in the transgenic animal of the present application is inherited by the offspring.

A F0 as a first-generation animal has an artificially modified myostatin gene. The F0 can produce progeny F1. The myostatin gene included in the F1 and sub-F1 progeny has the same nucleotide sequence as the artificially modified myostatin gene. The term “transgenic animal” in the present application includes the F0, F1, and sub-F1 progeny. In other words, even when direct artificial manipulation for transformation is not applied during the production process of animal F1 or after animal F1 is produced when animal F1 has a modified myostatin gene, it is a transgenic animal of the present application.

### Animal

An animal of the present application includes a non-human animal.

The animal includes mammals.

The mammals include ungulates.

The ungulates include artiodactyls. The artiodactyls may include pigs, deer, cattle, sheep, and goats, but are not limited thereto.

The mammals may include rodents. The rodents may include mice and rats, but are not limited thereto.

### Target Region

The term “target region” in the present application means a region in the genome of a wild type in which genes are to be artificially manipulated in order to produce a transgenic animal, and comprises a region including a protospacer sequence and a target sequence as indicated below.

### Protospacer Sequence

The term “protospacer sequence” in the present application refers to the 20 sequences adjacent to the PAM sequence by location of the PAM sequence in the target region of the present application. The protospacer sequence and the target sequence are complementary sequences. That is, this means the same sequence as the guide sequence that binds complementarily to the target sequence. However, the guide sequence may have a sequence which T (thymine) is replaced with U (uracil) in the protospacer sequence.

### Target Sequence

The term “target sequence” of the present application is a sequence included in the target region of the present application and is a sequence that complementarily binds to a protospacer sequence. The target sequence may complementarily bind with the guide sequence.

### Meaning of A, T, C, G, and U

As used herein, the symbols A, T, C, G, and U are interpreted as meanings understood by those of ordinary skilled in the art. Each of these symbols may be properly interpreted as a base, a nucleoside, or a nucleotide on DNA or RNA according to context and technology. For example, when each of the symbols means a base, the symbols A, T, C, G, and U can be interpreted as adenine (A), thymine (T), cytosine (C), guanine (G), or uracil (U), respectively. When each of the symbols means a nucleoside, the symbols A, T, C, G, and U can be interpreted as adenosine (A), thymine (T), cytidine (C), guanosine (G) or uridine (U), respectively. When meaning a nucleotide in the sequence, the symbols A, T, C, G, and U denote nucleotides including the nucleosides, respectively.

The following describes the present application in detail.

The present application relates to a transgenic animal having an artificially modified myostatin gene in which 12 base pairs of the second exon are deleted.

### Myostatin

The transgenic animal of the present application is characterized in that it contains artificially modified a myostatin gene.

The structure and function of myostatin will be described in detail below.

### Structure of Myostatin

The myostatin gene in higher organisms known to date is characterized by having three exons and two introns. It is known that the myostatin gene is mostly present in the muscle.

Myostatin mRNA produces myostatin protein, which is composed of approximately 375 amino acids and is divided into three parts: a signal peptide region, a propeptide (prodomain) region (28 kDa, N-terminus), and a mature region (12 kDa, C-terminus).

The structure of pro-myostatin, a precursor protein, is two identical subunits, and the mature regions form disulfide bonds with each other, thereby maintaining the form of a homodimeric protein.

### Myostatin Mature Protein Function and Signaling Pathway

As for the signaling pathway of a myostatin protein, after a first cleavage of a precursor protein, pro-myostatin, by an enzyme furin, it is divided into a propeptide region and a mature region. After cleavage, in a latent complex, the propeptide region binds to a mature region through non-covalent bonds. Then, as it is secreted out of a cell after a second cleavage is performed by BMP/Tolloid, the myostatin mature region is phosphorylated by binding to activin type II receptors. The signal is transmitted to the activin type I receptor again, and the signal is transmitted to the receptor-regulated proteins, Smad 2 and Smad 3, and Smed 2 and Smed 3 combine with co-Smad 4 to regulate the transcription of target genes. As a result of this signaling pathway, the mature myostatin protein is expressed.

### Conventional Modification of Myostatin Gene

In conventional studies related to a myostatin gene, studies in which a mature myostatin protein is not expressed have been conducted. By suppressing the expression of the mature myostatin protein, a study was conducted to prepare an animal in which a large amount of muscle was produced, and fat was reduced. In addition, research on the signaling pathway of myostatin is being conducted in the direction of utilizing it for diseases in which muscle mass is rapidly reduced, such as terminal cancer patients, and muscle fiber regeneration through studies in which mature myostatin protein is not expressed.

In many cases of myostatin transgenic animals, the myostatin gene is modified so that the myostatin protein which inhibits muscle growth, is not expressed much in somatic cells. In other words, it is a form in which expression of the mature myostatin protein is suppressed by modifying the cleavage region in the signaling pathway of the mature myostatin protein described above. Animals cloned by nuclear transfer of the somatic cells have a double muscle mass with increased muscle mass compared to wild-type animals.

However, a transgenic animal having modification of myostatin gene, which is obtained by the above conventional method has a short lifespan. Therefore, there is a disadvantage in that problem in reproduction and fatal side effects on health occur, especially in large animals.

The present application relates to a transgenic animal that minimizes side effects caused by conventional myostatin gene modification and emphasizes the advantages of myostatin gene modification.

Specifically, by targeting a specific region of exon 2 to delete 12 base pairs, where is not the cleavage region of a mature myostatin protein's signaling pathway, the present invention provides an animal with suppressed expression of the mature myostatin protein, compared to wild-type, rather than no expression.

### Myostatin Transgenic Animal

One aspect of the present application is a myostatin transgenic animal having an artificially modified myostatin gene. In one embodiment, it may be an ungulate animal, for example, a bovine.

In the following, the present disclosure will be described in detail, taking the bovine (cattle) having the artificially modified myostatin gene of the present application as an example.

### Characteristic 1—Genetic Modification of the Genome of Transgenic Animals

The transgenic animal of the present application may have a myostatin gene composition different from that of wild-type animals in terms of myostatin gene composition.

The genetic modification of the present application refers to a deletion of a nucleic acid sequence encoding four amino acid sequences (amino acids in the order of leucine, tryptophan, isoleucine, and tyrosine) of a specific conserved region among the amino acid sequences of myostatin protein.

The transgenic animal of the present application has a myostatin gene in which 12 base pairs of the second exon, which is a nucleic acid sequence encoding the amino acid sequence of the conserved region, is deleted.

When the transgenic animal is bovine, pig, or human, a deletion of the 12 base pairs may be a deletion of the base pairs at positions 93 to 104 of the sequence encoding the second exon of the myostatin gene of the wild-type (sequenced as 5′ to 3′).

When the transgenic animal is a mouse, a deletion of 12 base pairs may be a deletion of base pairs at positions 94 to 105 of the sequence encoding the second exon of the wild-type myostatin gene.

Characteristic 2—Changes in the mRNA Composition and Expression Level of the Myostatin Gene in Transgenic Animals

Transgenic animals of the present application may have different aspects of myostatin mRNA from wild-type animals. The transgenic animal of the present application has myostatin mRNA with 12 bases deleted.

In one embodiment of the present application, the amount of myostatin mRNA expression in transgenic animals can be measured.

In a specific embodiment, the amount of myostatin mRNA expression in the transgenic animal of the present application is at least 60% lower than that of the wild-type animal. Preferably, the amount of myostatin mRNA expression in the transgenic animal of the present application is less than that of the wild-type animal, but it does not mean that the expression is not absent.

### Characteristic 3—Changes in Protein Composition of Myostatin Gene in Transgenic Animals and Expression of Mature Myostatin Protein

A deleted 12 base pairs of a transgenic animal of the present application are a nucleic acid encoding a conserved amino acid sequence without a modification in an amino acid sequence for each species during the evolution of a myostatin gene. The conserved amino acid sequence is an amino acid sequence in the order of leucine, tryptophan, isoleucine, and tyrosine.

Therefore, a transgenic animal of the present application expresses a myostatin protein in which four amino acids in the sequence of leucine, tryptophan, isoleucine, and tyrosine are deleted compared to wild-type pro-myostatin protein.

A pro-myostatin protein in which the four amino acids are deleted may be one of SEQ ID NOs: 30 to 33.

A pro-myostatin protein of the transgenic animal may have modifications in some sequences but may have 90% or more homology with one of SEQ ID NOs: 30 to 33.

For example, when the transgenic animal is a bovine (cattle), a pro-myostatin protein of SEQ ID NO: 30 in which 4 amino acids are deleted, can be expressed.

For example, when the transgenic animal is a pig, a pro-myostatin protein of SEQ ID NO: 31 in which 4 amino acids are deleted can be expressed.

For example, when the transgenic animal is a mouse, a pro-myostatin protein of SEQ ID NO: 32 in which 4 amino acids are deleted, can be expressed.

For example, when the transgenic animal is a human, a pro-myostatin protein of SEQ ID NO: 33 in which 4 amino acids are deleted, can be expressed.

TABLE 1

| SEQ | |
|---|---|
| NO: | |
| ID | Pro-myostatin protein sequence |
| 30 | MQKLQISVYIYLFMLIVAGPVDLNENSEQKENVEKEGLCNACLWRENTTSSRLEAIKIQ |
| | ILSKLRLETAPNISKDAIRQLLPKAPPLLELIDQFDVQRDASSDGSLEDDDYHARTETV |
| | ITMPTESDLLTQVEGKPKCCFFKFSSKIQYNKLVKAQLRPVKTPATVFVQILRLIKPMK |
| | DGTRYTGIRSLKLDMNPGTGIWQSIDVKTVLQNWLKQPESNLGIEIKALDENGHDLAVT |
| | FPEPGEDGLTPFLEVKVTDTPKRSRRDFGLDCDEHSTESRCCRYPLTVDFEAFGWDWII |
| | APKRYKANYCSGECEFVFLQKYPHTHLVHQANPRGSAGPCCTPTKMSPINMLYENGEGQ |
| | IIYGKIPAMVVDRCGCS |
| 31 | MQKLQIYVYIYLFMLIVAGPVDLNENSEQKENVEKEGLCNACMWRQNTKSSRLEAIKIQ |
| | ILSKLRLETAPNISKDAIRQLLPKAPPLRELIDQYDVQRDDSSDGSLEDDDYHATTETI |
| | ITMPTESDLLMQVEGKPKCCFFKFSSKIQYNKVVKAQLRPVKTPTTVFVQILRLIKPMK |
| | DGTRYTGIRSLKLDMNPGTGIWQSIDVKTVLQNWLKQPESNLGIEIKALDENGHDLAVT |
| | FPGPGEDGLNPFLEVKVTDTPKRSRRDFGLDCDEHSTESRCCRYPLTVDFEAFGWDWII |
| | APKRYKANYCSGECEFVFLQKYPHTHLVHQANPRGSAGPCCTPTKMSPINMLYFNGKEQ |
| | IIYGKIPAMVVDRCGCS |
| 32 | MMQKLQMYVYIYLFMLIAAGPVDLNEGSEREENVEKEGLCNACAWRQNTRYSRIEAIKI |
| | QILSKLRLETAPNISKDAIRQLLPRAPPLRELIDQYDVQRDDSSDGSLEDDDYHATTET |
| | IITMPTESDFLMQADGKPKCCFFKFSSKIQYNKVVKAQLRPVKTPTTVFVQILRLIKPM |
| | KDGTRYTGIRSLKLDMSPGTGIWQSIDVKTVLQNWLKQPESNLGIEIKALDENGHDLAV |
| | TFPGPGEDGLNPFLEVKVTDTPKRSRRDFGLDCDEHSTESRCCRYPLTVDFEAFGWDWI |
| | IAPKGYKANYCSGECEFVFLQKYPHTHLVHQANPRGSAGPCCTPTKMSPINMLYENGKE |
| | QIIYGKIPAMVVDRCGCS |
| 33 | MQKLQLCVYIYLFMLIVAGPVDLNENSEQKENVEKEGLCNACTWRQNTKSSRIEAIKIQ |
| | ILSKLRLETAPNISKDVIRQLLPKAPPLRELIDQYDVQRDDSSDGSLEDDDYHATTETI |
| | ITMPTESDFLMQVDGKPKCCFFKFSSKIQYNKVVKAQLRPVETPTTVFVQILRLIKPMK |
| | DGTRYTGIRSLKLDMNPGTGIWQSIDVKTVLQNWLKQPESNLGIEIKALDENGHDLAVT |
| | FPGPGEDGLNPFLEVKVTDTPKRSRRDFGLDCDEHSTESRCCRYPLTVDFEAFGWDWII |
| | APKRYKANYCSGECEFVFLQKYPHTHLVHQANPRGSAGPCCTPTKMSPINMLYENGKEQ |
| | IIYGKIPAMVVDRCGCS |

The four amino acids to be deleted do not overlap with a region of a pro-myostatin protein that is cleaved in the process of forming a mature myostatin protein.

Since a site of the amino acid deletion is not included in a region where cleavage occurs, the pro-myostatin protein becomes a mature myostatin protein through a normal signaling process. That is, a deletion of a specific amino acid in the present application does not affect the formation process of a normal mature myostatin protein.

Therefore, a mature myostatin protein expressed by a myostatin transgenic animal of the present application is the same as that of the wild-type. That is, it is characterized by being identical to an amino acid sequence of a wild-type mature myostatin protein.

In one embodiment, the mature myostatin protein of the transgenic animal may be one of SEQ ID NOs: 34 to 37.

A mature myostatin protein of the transgenic animal may have modifications in some sequences but may have 90% or more homology with one of SEQ ID NOs: 34 to 37.

For example, when the transgenic animal is a bovine (cattle), a mature myostatin protein of SEQ ID NO: 34 identical to that of a wild-type bovine can be expressed.

For example, when the transgenic animal is a pig, a mature myostatin protein of SEQ ID NO: 35 identical to that of a wild-type pig can be expressed.

For example, when the transgenic animal is a mouse, a mature myostatin protein of SEQ ID NO: 36 identical to that of a wild-type mouse can be expressed.

For example, when the transgenic animal is a human, a mature myostatin protein of SEQ ID NO: 37 identical to that of a wild-type human can be expressed.

TABLE 2

| SEQ | |
|---|---|
| NO: | |
| ID | Mature myostatin protein sequence |
| 34 | FGLDCDEHSTESRCCRYPLTVDFEAFGWDWIIAPKRYKANYCS |
| | GECEFVFLQKYPHTHLVHQANPRGSAGPCCTPTKMSPINMLYE |
| | NGEGQIIYGKIPAMVVDRCGCS |
| 35 | CCRYPLTVDFEAFGWDWIIAPKRYKANYCSGECEFVFLQKYPH |
| | THLVHQANPRGSAGPCCTPTKMSPINMLYFNGKEQIIYGKIPA |
| | MVVDRCGCS |
| 36 | CCRYPLTVDFEAFGWDWIIAPKGYKANYCSGECEFVFLQKYPH |
| | THLVHQANPRGSAGPCCTPTKMSPINMLYFNGKEQIIYGKIPA |
| | MVVDRCGCS |
| 37 | CCRYPLTVDFEAFGWDWIIAPKRYKANYCSGECEFVFLQKYPH |
| | THLVHQANPRGSAGPCCTPTKMSPINMLYFNGKEQIIYGKIPA |
| | MVVDRCGCS |

A mature myostatin protein may have a monomeric or dimeric form in the blood. The transgenic animal of the present application can express the same mature myostatin protein as the wild-type. That is, the mature myostatin protein of the transgenic animal of the present application has the same amino acid sequence as the mature myostatin protein of a wild-type animal.

In one embodiment of the present application, a mature myostatin protein of the transgenic animal can be compared and identified with a wild-type mature myostatin protein through mass spectrometry.

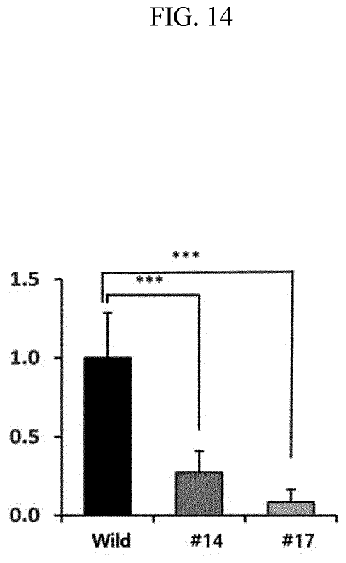

In one embodiment of the present application, an expression level of mature myostatin protein in the transgenic animal of the present application may be lower than that of a wild-type animal. This result can also be seen from the fact that the amount of myostatin mRNA expression in the transgenic animal of the present application was reduced compared to the amount of myostatin mRNA expression in a wild-type (see FIG. 14).

### Characteristic 4—Increased Muscle Mass

The transgenic animals of the present disclosure exhibit a muscularized phenotype due to reduced expression of myostatin mRNA and mature myostatin protein compared to wild-type animals. The muscularized phenotype refers to phenotypes such as increased muscle mass, increased number of muscle cells, increased size of muscle cells, and increased rate of muscle cell differentiation.

In specific embodiments, a transgenic animal of the present disclosure may have at least a 1%, 5%, 10%, 15%, 20%, 25%, 30%, 35%, or 40% increase in muscle mass compared to a wild-type animal.

### Characteristic 5—No Side Effects Such as Shortened Life Span

It is known that conventional myostatin transgenic animals are associated with shortened lifespans and health abnormalities.

Unlike conventional myostatin transgenic animals, the transgenic animal of the present application is not no-expressing myostatin mRNA and mature myostatin protein. In other words, the expression of myostatin mRNA and mature myostatin protein is reduced compared to wild-type animals that do not express myostatin mRNA and mature myostatin protein.

Thus, unlike the shortened life span and health abnormalities that can result from not expressing myostatin mRNA and mature myostatin protein, there may be no abnormalities in health.

In a specific embodiment, bovine (cattle) having a myostatin gene in which 12 base pairs are deleted of the present application were found to be healthy and free of abnormalities in health.

In one embodiment, a bovine having a myostatin gene in which 12 base pairs are deleted of the present application can breed offspring through reproduction.

### Myostatin Transgenic Cells

Another aspect of the present application is an engineered cell having an artificially modified myostatin gene.

The engineered cell may be embryonic cells, somatic cells, or stem cells.

In certain embodiments, the cells include but are not limited to, for example, ovocytes, epithelial cells, fibroblasts, nerve cells, keratinocytes, hematopoietic cells, melanocytes, chondrocytes, macrophages, monocytes, muscle cells, and B lymphocytes, T lymphocytes, embryonic stem cells, embryonic germ cells, fetal-derived cells, placental cells, and embryonic cells. In addition, adult stem cells derived from various tissues of origin can be used, for example, tissue-derived stem cells from fat, uterus, bone marrow, muscle, placenta, cord blood, or skin (epithelium). Non-human host embryos can generally be embryos containing a 2-cell stage, a 4-cell stage, an 8-cell stage, a 16-cell stage, a 32-cell stage, a 64-cell stage, an aborted embryo, or a blastocyst.

The characteristics of the engineered cell of the present application are the same as the characteristics 1 to 3 of the transgenic animal above.

In summary, the engineered cell has a myostatin gene in which 12 base pairs of the second exon are deleted.

The genetic modification of the engineered cell refers to a deletion of a nucleic acid sequence encoding four amino acid sequences (amino acids in the order of leucine, tryptophan, isoleucine, and tyrosine) of a specific conserved region among the amino acid sequences of myostatin protein. Therefore, the engineered cell has a myostatin gene in which 12 base pairs of the second exon, which is a nucleic acid sequence encoding the amino acid sequence of the conserved region, is deleted.

The engineered cell may have a different aspect of myostatin mRNA from wild-type cells. The engineered cell of the present application has myostatin mRNA with 12 bases deleted.

The amount of myostatin mRNA expression in the engineered cells is lower than that of wild-type animal's cells.

A prepro-myostatin protein must go through a cleavage step to become an active state mature myostatin protein.

Since the locations of the 4 amino acids deleted in the transgenic cells are not included in the region where cleavage occurs, the pro-myostatin protein becomes a mature myostatin protein through a normal signaling process. That is, the deletion of a specific amino acid in the present application does not affect the formation process of a normal mature myostatin protein.

That is, a mature myostatin protein expressed by the engineered cell in which 12 base pairs of a myostatin gene of the present application are deleted has the same amino acid sequence as a mature myostatin protein of wild-type cell.

### Composition for Genetic Manipulation

According to another aspect of the disclosure provided in the present application, a composition for genetic manipulation that modifies the myostatin gene is provided.

To modify the myostatin gene, the composition for gene manipulation may include:
a guide RNA comprising a guide sequence that forms a complementary bond with a target sequence of a myostatin gene or a DNA encoding the guide RNA; and
a Cas protein or a nucleic acid sequence encoding the Cas protein.

The target sequence is a sequence complementary to the protospacer sequence that is targeted by the composition and is included within a target region.

The target sequence is located in the second exon (Exon 2) of the myostatin gene.

### Target Sequence

A composition of the present application targets the myostatin gene in order to modify the myostatin gene.

A portion that can be targeted by the composition is referred to as a target region.

The target region is located in the second exon (Exon 2) of the myostatin gene.

The target region includes a target sequence and a protospacer sequence, and a sequence to which a guide sequence of the composition complementarily binds is called a target sequence.

In the present application, since there is a genetic sequence difference between species, it may be easy to target a nucleic acid encoding a conserved region of an amino acid sequence of myostatin protein as a target region of a composition for genetic manipulation.

Accordingly, the target sequence is configured to include some or all of a sequence encoding a conserved amino acid sequence of myostatin protein by species, as described below.

In the following, a conserved amino acid sequences of pro-myostatin proteins by species are described in detail. In one embodiment, the conserved amino acid sequence is described relative to bovine versus human, pig, or mouse. Animals having the conserved amino acid sequence are not limited thereto.

A comparison of the pro-myostatin protein sequences of bovine, human, pig, and mouse is provided in part below [Table 3]. An amino acid sequences at positions 156 to 159 of each pro-myostatin protein are conserved, and the following a conserved amino acid sequence are in order of leucine, tryptophan, isoleucine, and tyrosine (see the bolded column in the table below).

TABLE 3

| | 152 | 153 | 154 | 155 | 156 | 157 | 158 | 159 | 160 | 161 | 162 | 163 | 164 | 165 |
|---|---|---|---|---|---|---|---|---|---|---|---|---|---|---|
| Bovine | V | K | A | Q | L | W | I | Y | L | R | P | V | E | T |
| Human | ″ | ″ | ″ | ″ | ″ | ″ | ″ | ″ | ″ | ″ | ″ | ″ | K | ″ |
| Pig | ″ | ″ | ″ | ″ | ″ | ″ | ″ | ″ | ″ | ″ | ″ | ″ | K | ″ |

| | 153 | 154 | 155 | 156 | 157 | 158 | 159 | 160 | 161 | 162 | 163 | 164 | 165 | 166 |
|---|---|---|---|---|---|---|---|---|---|---|---|---|---|---|
| Mice | V | K | A | Q | L | W | I | Y | L | R | P | V | K | T |

Location of deletion of a specific amino acid of a pro-myostatin protein of the present application is such a conserved amino acid sequence, that is, a 156th to 159th amino acid sequence of a myostatin protein.

These amino acid sequences are located at positions 157th to 160th of a myostatin protein in mice, but the amino acid sequences in mice are the same as leucine, tryptophan, isoleucine, and tyrosine.

A region targeted by a composition in the present application may comprises some or all of a region encoding the conserved amino acid sequence. A target sequence can be designed around one strand of a DNA double strands including the conserved region.

The target sequence may comprise some or all of the sequences of 5′-CTT-3′, 5′-CTC-3′, 5′-CTA-3′, or 5′-CTG-3′ encoding leucine in the amino acid sequence, 5′-TGG-3′ encoding tryptophan of amino acid sequence, 5′-ATT-3′, 5′-ATC-3′, or 5′-ATA-3′ encoding isoleucine of amino acid sequence, 5′-TAT-3′ or 5′-TAC-3′ encoding tyrosine of amino acid sequence, or some or all of the complementary sequences of such sequences. In one embodiment of the present application, the target sequence may comprise SEQ ID NO: 28-5′-ATATATCCACAG-3′.

In another embodiment of the present application, the target sequence may comprise SEQ ID NO: 29-5′-CTGTGGATATAT-3′.

In order to design a target sequence, a PAM sequence in the target region should be considered. The PAM sequence may differ depending on an origin of a Cas protein.

The PAM sequence and the sequence adjacent thereto are referred to as protospacer sequences. The protospacer sequence is composed of 20 or less base sequences, except for the PAM sequence. The protospacer sequence and the target sequence are complementary sequences.

In one example, a target sequence of a myostatin gene may be selected from SEQ ID NOs: 38 to 60 in [Table 4].

For example, SEQ ID NOS: 38 to 43 may be a target sequence of a myostatin gene of a bovine.

For example, SEQ ID NO: 42, SEQ ID NO: 43, and SEQ ID NOs: 45 to 48 may be a target sequence of a myostatin gene of a pig.

For example, SEQ ID NOs: 49 to 55 may be a target sequence of a myostatin gene of a human.

For example, SEQ ID NOs: 56 to 60 may be a target sequence of a myostatin gene of a mouse.

TABLE 4
Target sequence

CGGAGTCTATATAGGTGTCA (SEQ ID NO: 38)
GGAGTCTATATAGGTGTCAA (SEQ ID NO: 39)
GGGTTGACACCTATATAGAC (SEQ ID NO: 40)
CCGGAGTCTATATAGGTGTC (SEQ ID NO: 41)
TCCGGGTTGACACCTATATA (SEQ ID NO: 42)
CTATATAGGTGTCAACCCGG (SEQ ID NO: 43)
GTGTCAACCCGGAAATGATC (SEQ ID NO: 44)
CAGAGTCTATATAGGTGTCA (SEQ ID NO: 45)
AGAGTCTATATAGGTGTCAA (SEQ ID NO: 46)
CCAGAGTCTATATAGGTGTC (SEQ ID NO: 47)
GTGTCAACCCGGAAATGATG (SEQ ID NO: 48)
CAGAGTTTATATAGGTATCA (SEQ ID NO: 49)
AGAGTTTATATAGGTATCAA (SEQ ID NO: 50)
TACCTATATAAACTCTGGGC (SEQ ID NO: 51)
TCCGGGTTGATACCTATATA (SEQ ID NO: 52)
CCAGAGTTTATATAGGTATC (SEQ ID NO: 53)
TTATATAGGTATCAACCCGG (SEQ ID NO: 54)
GTATCAACCCGGAAATGATG (SEQ ID NO: 55)
CAGACTCTATATAGGTGTCA (SEQ ID NO: 56)
AGACTCTATATAGGTGTCAA (SEQ ID NO: 57)
CCAGACTCTATATAGGTGTC (SEQ ID NO: 58)
TCTATATAGGTGTCAACCCG (SEQ ID NO: 59)
GTGACAACCCGAAAATGATG (SEQ ID NO: 60)

In one embodiment, the composition of the present application comprises a guide RNA comprising a guide sequence complementary to a target sequence or a DNA encoding the guide RNA; and a CAS protein or a nucleic acid sequence encoding the CAS protein.

### Guide RNA or DNA Encoding the Guide RNA

A guide RNA of the present application comprises a guide sequence complementary to the target sequence described above.

The guide RNA may comprise a first sequence, which is a guide sequence that can complementarily bind to the target sequence, and a second sequence involved in forming a complex by interacting with a Cas protein.

The first sequence of the guide RNA of the present application is the same sequence as the protospacer sequence, which is complementary to the designed target sequence, and is an RNA sequence composed of U (uracil) instead of T (thymine) among the protospacer sequences.

In another aspect, the first sequence of the present application may be part of crRNA, and the second sequence may include another part of crRNA and/or tracrRNA. As an example, the guide RNA may be first and second sequences composed only of crRNA, and as another example, the guide RNA may be first and second sequences including crRNA and tracrRNA.

In this case, the first sequence may be determined according to the target sequence, and a part of the second sequence may be determined according to the type of Cas protein-derived microorganism.

For example, in the case of a guide RNA that binds to a Cas protein derived from Streptococcus pyogenes, the first sequence may be a part of a crRNA sequence, and the second sequence may include tracrRNA.

In one embodiment, in the case of guide RNA that binds a Streptococcus pyogenes protein, the second sequence may include

(SEQ ID NO: 61)
5′GUUUUAGUCCCUGAAAAGGGACUAAAAUAAAGAGUUUGC
GGGACUCUGCGGGGUUACAAUCCCCUAAAACCGCUUUU-3′.

Meanwhile, the guide RNA of the present application may be in the form of a single sequence in which the first sequence and the second sequence are linked. Alternatively, the guide RNA may be composed of two separate sequences consisting of a sequence including the first sequence and a sequence including a part of the second sequence, which may be composed of crRNA and tracrRNA, respectively.

Hereinafter, examples of a guide sequence that can be used in one embodiment of the present application are shown in a table. The guide sequence listed in [Table 5] are RNA sequences that can complementarily bind to a target sequence of a myostatin gene.

The guide sequence listed in Table 5 are a guide sequence capable of targeting a sequence in Table 2, respectively.

The guide sequence of the present application may be a sequence selected from SEQ ID NO: 62 to SEQ ID NO: 84.

For example, SEQ ID NOs: 62 to 68 are guide sequences capable of complementarily binding to a target sequence of a myostatin gene of a bovine.

For example, SEQ ID NO: 66, SEQ ID NO: 67, and SEQ ID NO: 69 to SEQ ID NO: 72 are guide sequences capable of complementarily binding to a target sequence of a myostatin gene of a pig.

For example, SEQ ID NOs: 73 to 79 are guide sequences capable of complementarily binding to a target sequence of a myostatin gene of a human. For example, SEQ ID NO: 80 to SEQ ID NO: 84 are guide sequences capable of complementary binding to a target sequence of a myostatin gene of a mouse.

In one embodiment of the present disclosure, a form of a complex of a guide RNA comprising the above guide sequence and a Cas protein (Ribonucleoprotein particle: RNP) can be injected into a cell or embryo.

TABLE 5

Guide sequence

5′-GCCUCAGAUAUAUCCACAGU-3′ (SEQ ID NO: 62)
5′-CCUCAGAUAUAUCCACAGUU-3′ (SEQ ID NO: 63)
5′-CCCAACUGUGGAUAUAUCUG-3′ (SEQ ID NO: 64)
5′-GGCCUCAGAUAUAUCCACAG-3′ (SEQ ID NO: 65)
5′-AGGCCCAACUGUGGAUAUAU-3′ (SEQ ID NO: 66)
5′-GAUAUAUCCACAGUUGGGCC-3′ (SEQ ID NO: 67)
5′-CACAGUUGGGCCUUUACUAG-3′ (SEQ ID NO: 68)
5′-GUCUCAGAUAUAUCCACAGU-3′ (SEQ ID NO: 69)
5′-UCUCAGAUAUAUCCACAGUU-3′ (SEQ ID NO: 70)
5′-GGUCUCAGAUAUAUCCACAG-3′ (SEQ ID NO: 71)
5′-CACAGUUGGGCCUUUACUAC-3′ (SEQ ID NO: 72)
5′-GUCUCAAAUAUAUCCAUAGU-3′ (SEQ ID NO: 73)
5′-UCUCAAAUAUAUCCAUAGUU-3′ (SEQ ID NO: 74)
5′-AUGGAUAUAUUUGAGACCCG-3′ (SEQ ID NO: 75)
5′-AGGCCCAACUAUGGAUAUAU-3′ (SEQ ID NO: 76)
5′-GGUCUCAAAUAUAUCCAUAG-3′ (SEQ ID NO: 77)
5′-AAUAUAUCCAUAGUUGGGCC-3′ (SEQ ID NO: 78)
5′-CAUAGUUGGGCCUUUACUAC-3′ (SEQ ID NO: 79)
5′-GUCUGAGAUAUAUCCACAGU-3′ (SEQ ID NO: 80)
5′-UCUGAGAUAUAUCCACAGUU-3′ (SEQ ID NO: 81)
5′-GGUCUGAGAUAUAUCCACAG-3′ (SEQ ID NO: 82)
5′-AGAUAUAUCCACAGUUGGGC-3′ (SEQ ID NO: 83)
5′-CACAGUUGGGCUUUUACUAC-3′ (SEQ ID NO: 84)

Meanwhile, in another aspect, the present application may provide DNA encoding the guide RNA. In this case, a DNA sequence encoding the guide RNA is a sequence encoding a first sequence, the guide sequence, and comprises a DNA sequence identical to a target sequence represented by SEQ ID NOs: 38 to 60, respectively; and a DNA sequence encoding the second sequence.

### Cas Protein or Nucleic Acid Encoding Cas Protein

The Cas protein of the present application may be selected from the group consisting of a Cas9 protein derived from Streptococcus pyogenes, a Cas9 protein derived from Campylobacter jejuni, a Cas9 protein derived from Streptococcus thermophilus, a Cas9 protein derived from Staphylococcus aureus, a Cas9 protein derived from Neisseria meningitidis, and Cas12a (Cpf1) protein. In the present application, the Cas protein may be a wild-type or a mutant form thereof.

In the present application, the Cas protein or the nucleic acid encoding the Cas protein may further include an element commonly used for delivery into the nucleus of eukaryotic cells, for example, a nuclear localization sequence (NLS).

In one embodiment, the Cas protein may be a Cas9 protein derived from Streptococcus pyogenes, a Cas9 protein derived from Staphylococcus aureus, or a Cas12a (Cpf1) protein.

The PAM sequence may vary depending on the Cas protein. In one embodiment, SpCas9 has a PAM sequence of NGG. In one embodiment, SaCas9 has a PAM sequence of NNGRR or NNGRRT. In one embodiment, Cas12a (Cpf1) has a PAM sequence of TTTN. The N is any one of A, T, G or C. The R is A or G.

### Form of Composition for Genetic Manipulation

A composition for genetic manipulation of myostatin of the present application may include: a guide RNA or a nucleic acid encoding the guide RNA; and a Cas protein or a nucleic acid encoding the Cas protein, each independently or together.

A guide RNA of the present application may be delivered into a cell in the form of RNA or DNA encoding the guide RNA. The guide RNA may be in the form of an independent RNA, RNA included in a viral vector, or encoded in a vector.

The Cas protein of the present application can be delivered into cells in the form of RNA or DNA encoding the RNA. The Cas protein may be in the form of an independent RNA, RNA included in a viral vector, or encoded in a vector.

At this time, the viral vector may be selected from the group consisting of a retrovirus vector, a lentivirus vector, an adenovirus vector, an adeno-associated virus (AAV) vector, a vaccinia virus vector, a poxvirus vector, and a herpes simplex virus vector.

In one embodiment, the guide RNA and the Cas protein may be configured in the form of plasmid DNA including a sequence encoding each RNA and a promoter, and plasmid DNA including a sequence encoding a protein and a promoter.

In another embodiment, the guide RNA and the Cas protein may be configured in a form including a sequence encoding a RNA or protein and a promoter in one plasmid DNA.

As another form, the guide RNA and the Cas protein may be configured in the form of a viral vector rather than plasmid DNA.

In another embodiment, the guide RNA and the Cas protein may be configured in the form of mRNA. At this time, the guide RNA may be prepared by in vitro transcription using any in vitro transcription system known in the art.

The guide RNA and Cas protein of the present application may preferably be configured in the form of a ribonucleoprotein (RNP) complex in which the guide RNA and Cas protein are bound.

In another embodiment, the guide RNA and the Cas protein may be configured in different forms. For example, the guide RNA may be configured in the form of an independent RNA, and the Cas protein may be configured in the form of a vector including a sequence encoding the protein and a promoter.

In addition, the composition may be configured in various forms. Therefore, since those skilled in the art can appropriately use methods known in the art, there is no limitation.

### Method for Producing an Engineered Cell Comprising an Artificially Modified Myostatin Gene

Another aspect of the disclosure provided in the present application relates to a method for producing an engineered cell having a myostatin gene in which 12 base pairs of the second exon are deleted using the above composition.

The cells may be embryonic cells, somatic cells, or stem cells.

In certain embodiments, the cells include but are not limited to, for example, oocytes, epithelial cells, fibroblasts, nerve cells, keratinocytes, hematopoietic cells, melanocytes, chondrocytes, macrophages, monocytes, muscle cells, and B lymphocytes, T lymphocytes, embryonic stem cells, embryonic germ cells, fetal-derived cells, placental cells and embryonic cells. In addition, adult stem cells derived from various tissues of origin can be used, for example, tissue-derived stem cells from fat, uterus, bone marrow, muscle, placenta, cord blood, or skin (epithelium). Non-human host embryos can generally be embryos containing a 2-cell stage, a 4-cell stage, an 8-cell stage, a 16-cell stage, a 32-cell stage, a 64-cell stage, an aborted embryo, or a blastocyst.

Preferably, the cells may be embryonic cells.

The cells may be derived from animals.

The animal includes mammals.

The mammals include ungulates.

The ungulates may include bovine and pigs but are not limited thereto.

The mammals include rodents.

The rodents may include a mouse but are not limited thereto.

The method for producing an engineered cell comprising an artificially modified myostatin gene of the present application,

may include contacting a cell with the composition. The contacting step may be performed in vivo or ex vivo.

For example but is not limited to, the contacting step may be introduced into cells by transient transfection, microinjection, transduction, cell fusion, calcium phosphate precipitation, liposome-mediated transfection, DEAE Dextran-mediated transfection, polybrene-mediated transfection, electroporation, gene gun, and other known methods for introducing nucleic acids into the cell.

By the introduced composition, an indel (indel: insertion and deletion) occurs in the genome of the cell.

“Indel” is a general term for mutations in which some nucleotides are inserted or deleted in the middle of the nucleotide sequence of DNA. The indels may be introduced into the target sequence during the process of cutting and repairing the nucleic acid (DNA, RNA) by the guide RNA-CRISPR complex of the composition.

As a result of the indel, the engineered cell of the present application has a myostatin gene in which 12 base pairs in the second exon are deleted by the composition.

In addition, according to the method for producing an engineered cell comprising an artificially modified myostatin gene described above, the engineered cells of the present application have a genetic modification resulting in an in-frame deletion.

An effect of the in-frame deletion is described below.

Effect of in-Frame Deletion

An engineered cell having a myostatin gene in which 12 base pairs of the second exon of the present application are deleted has a genetic modification resulting in an in-frame deletion.

An ‘In-frame deletion’ requires a deletion of at least three DNA bases, usually a multiple of three, to result in the loss of the entire corresponding codon, which can lead to the deletion of the corresponding amino acid in the resulting protein.

Since the present application is characterized by a deletion of 12 bases of the myostatin gene, the form of the deletion is an in-frame deletion, and no frame shifted modification occurs.

As a result of the in-frame deletion, the protein is in the form of a protein in which four amino acids are deleted, and translation to other amino acids or alteration of stop codons, which can occur in general frame shift modifications, do not occur. That is, the remaining amino acids, except for the four amino acids, can normally be translated into proteins through transcription in the myostatin gene.

### Method for Producing Transgenic Animal Comprising an Artificially Modified Myostatin Gene

Another aspect of the disclosure provided in the present application relates to a method for producing an animal using the engineered cell. Specifically, the present application relates to a method for producing an animal having a myostatin gene in which 12 base pairs of the second exon are deleted.

In an arbitrary embodiment, the production method is performed by transferring an embryonic cell having a myostatin gene having 12 base pairs of the second exon deleted into a surrogate mother to produce a transgenic animal having an artificially modified myostatin gene having 12 base pairs of the second exon deleted.

In another embodiment, the production method relates to a method for producing an animal having a transgenic tissue or organ by injecting the composition into an animal tissue or organ.

The animal includes mammals.

The mammals include ungulates.

The ungulates may include bovine and pigs but are not limited thereto.

The mammals include rodents.

The rodents may include a mouse, but is not limited thereto.

### Method for Producing Transgenic Animal Using the Engineered Cell

The method for producing transgenic animal comprising an artificially modified myostatin gene of the present application,

in one embodiment, the method may comprise transferring the engineered cell generated in the above step to a surrogate mother, which has an artificially modified myostatin gene in which 12 base pairs of the second exon are deleted.

Conventional descriptions of each step can be understood by referring to methods for producing transgenic animals known in the art.

In the present application, an embryonic cell can be produced by the methods described in the “Method for producing an engineered cell comprising an artificially modified myostatin gene” method above, complete with the step of contacting the cell with the composition.

The embryonic cells may develop into blastocysts in an in vitro culture process.

An animal having a myostatin gene in which 12 base pairs of the second exon are deleted can be produced by transferring the blastocyst to a surrogate mother.

In one embodiment of the present disclosure, an embryo having an artificially modified myostatin gene in which 12 base pairs of the second exon are deleted is transplanted to produce an animal having an artificially modified myostatin gene in which 12 base pairs of the second exon are deleted, preferably a bovine (cattle) having a myostatin gene in which 12 base pairs of the second exon are deleted.

The transgenic animal may be a chimeric or homologous transgenic animal.

The method for producing transgenic animal comprising an artificially modified myostatin gene of the present application,
for another specific example, may comprise
obtaining the engineered somatic cell comprising an artificially modified myostatin gene described above; preparing an enucleated oocyte by removing a nucleus from an egg of an animal; microinjecting or a nucleus of the engineered somatic cell into the enucleated oocyte, and fusing; activating the fused egg; and transferring the activated egg into a surrogate mother.

Conventional descriptions of each step can be understood by referring to methods for preparing transgenic animals using conventional somatic cell nuclear transfer technology known in the art.

A transgenic animal can be prepared by transplanting a somatic cell or its nucleus, having an artificially modified myostatin gene according to the above method, into an enucleated oocyte using SCNT (somatic cell nuclear transfer) method. The transgenic animal may be a homologous transgenic animal.

In another embodiment, as a method for producing a homologous transgenic animal, a first transgenic animal having a myostatin gene in which 12 base pairs of the second exon are deleted, may be crossbreed with each other to produce the homologous transgenic animal.

The transgenic animal obtained through the crossing may include the same myostatin gene as the one in which 12 base pairs of the myostatin gene included in the animal genome of the first transgenic animal are deleted.

Method for Producing Animals in which Some Tissues are Transgenic

The transgenic animal of the present application may be an animal having an artificially modified myostatin gene in which 12 base pairs of the second exon are deleted in some tissues of the animal.

The tissue may be epithelial tissue, connective tissue, or muscle tissue, but preferably may be a muscle tissue including a myostatin gene.

The method may include the steps of introducing the composition described above into a tissue of an animal.

When the composition is introduced into an animal tissue, the animal can be tissue-specifically engineered to have a modified myostatin gene within the tissues.

The introduction may be performed by injection, implantation, or transplantation.

The introduction may be performed by a selected route of administration: subretinal, subcutaneously, intradermally, intraocularly, intravitreally, intratumorally, intranodal, intramedullary, intramuscularly, or
intraperitoneally.

### Use of Myostatin Transgenic Animals of the Present Application

### Improved Breed Animal

An animal having a myostatin gene in which 12 base pairs of the second exon are deleted can be used as an improved breed animal. The improved breed animal may be a bovine, pig, mouse, or rat in which 12 base pairs of the myostatin gene are deleted but are not limited thereto. The improved breed animal may be an improved breed animal having developed muscles compared to a wild-type animal. The improved breed animal may be an improved breed animal having reduced fat compared to a wild-type animal.

### Animals for Disease Model Research

Animals having the artificially modified myostatin gene in which 12 base pairs of the second exon are deleted can be used as disease model research animals. The disease model research animal may be a bovine, pig, mouse, or rat in which 12 base pairs of the myostatin gene are deleted but are not limited thereto. The disease model may be a study including muscle atrophy, sarcopenia, and myofibrillar reduction, but is not limited thereto.

### Disease Resistant Animals

An animal having a myostatin gene in which 12 base pairs of the second exon are deleted can be used as a disease resistant animal. The disease resistant animal may be a bovine, pig, mouse, or rat in which 12 base pairs of the myostatin gene are deleted but are not limited thereto. The disease may be a study including muscle atrophy, sarcopenia, and myofibrillar reduction, but is not limited thereto.

Use of by-Products

Flesh, organs, skin, fur, and body fluids of a transgenic animal having a myostatin gene in which 12 base pairs of the second exon are deleted may be used but are not limited thereto. The transgenic animal may be a bovine, pig, mouse, or rat in which 12 base pairs of the myostatin gene are deleted but are not limited thereto. Compared to wild-type animals, the transgenic animal may have a low-fat content and a high muscle content. Therefore, it is possible to obtain high-quality meat with low-fat content and high muscle content as a by-product of the animal.

### Use of Composition for Genetic Manipulation of the Present Application

Another aspect of the disclosure provided in the present application relates to the use of the composition for genetic manipulation of the present application.

The composition described above may be used for purposes of increasing muscle mass but is not limited thereto.

At this time, the subject to which the composition can be administered may be mammals including, primates such as humans and monkeys, rodents such as mice and rats, and ungulates such as bovine, pigs, and horses.

Another aspect of the disclosure provided in the present application may provide a method capable of increasing the muscle of the administered tissue, including the step of administering the composition for genetic manipulation of the present application.

The composition may be administered to a specific body location of a subject to whom the composition is administered.

The specific body location may be around a tissue requiring muscle growth.

The specific body location may be around a tissue in which muscles are not developed in a state of infancy.

The administration may be performed by injection, transfusion, implantation, or transplantation.

The administration may be performed by a selected route of administration: subcutaneously, intradermally, intramuscularly, or intraperitoneally.

A single dose (effective amount to achieve a predetermined desired effect) of the myostatin gene manipulation composition may be selected from any integer value within the above numerical ranges, such as, but not limited thereto, 10⁴ to 10⁹ cells per kilogram of body weight of the subject, such as 10⁵ to 10⁶ cells/kg (body weight), and may be appropriately administered taking into account the age, health, and weight of a subject.

When the myostatin gene is artificially manipulated by the method or composition of some embodiments disclosed in the present specification, an effect such as an increase in muscle may be obtained through this.

Hereinafter, the present application will be described in more detail through examples.

These embodiments are only intended to explain the present application in more detail, and it will be obvious to those of ordinary skilled in the technical field to which the present application belongs that the scope of the present application is not limited by these embodiments.

### EXAMPLE

### [Example 1] Single Guide RNA (sgRNA) Design

The sgRNA containing sequences complementary to every single strand of 12 base pairs of myostatin was designed by CHOPCHOP software (World Wide Web at chopchop.cbu.uib.no/). The sgRNA includes the above complementary binding sequence and is used among the PAM sequences of CRISPR/SpCas9, CRISPR/SaCas9, or CRISPR/Cpf1 for the myostatin gene. The sgRNA used in the experiment was designed to include at least one of the guide sequences in Table 5.

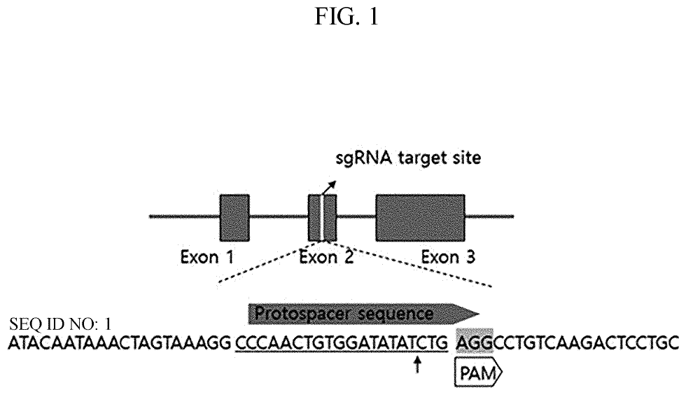

FIG. 1 shows one of protospacer sequence of the myostatin gene. In the sequence showed by FIG. 1., the underlined sequence corresponds to SEQ ID NO: 64 in which U (uracil) is replaced with T (thymine), among the guide sequences of Table 5.

By binding the guide RNA to the complementary sequence (target sequence) of the sequence through the sequence of FIG. 1, the binding sequence of the guide RNA may be predicted in the sequence.

### [Example 2] In Vitro Maturation of Oocytes

Ovaries were collected from local slaughterhouses and delivered to the laboratory within two hours. Ovaries transferred from the slaughterhouse were aspirated with an 18-gauge needle syringe to obtain a cumulus-oocyte complex (COC) from follicles with 2 to 8 mm in diameter. COCs were classified as surrounded by more than three layers of cumulus cells and evenly distributed cytoplasm. During the in vitro maturation process, COCs were cultured in a humidified atmosphere of 5% CO₂ at 38.5° C. in a chemically defined TCM-199 medium containing 0.005 AU/ml FSH (Antrin, Teikoku, Cat. No. F2293), 1 μg/ml 17ß-estradiol (Sigma-Aldrich, Cat. No. E4389), 100 μM cysteamine (Sigma-Aldrich, Cat. No. M6500), and 10% FBS (Gibco, Cat. No. GIB-16000-044).

### [Example 3] Sperm Purification, In Vitro Fertilization, and In Vitro Culture of Embryos

Motile sperm were purified by the Percoll gradient method. Sperm from semen thawed at 35° C. were filtered by centrifugation in a Percoll discontinuous gradient (45% to 90%) at 1500 rpm for 15 min. To create a 45% Percoll solution, add a 1 ml volume of TALP to 1 ml of 90% Percoll. The sperm pellet was centrifuged at 1500 rpm for 5 minutes and washed twice by adding 3 ml of TALP. Motile sperm purified through the Percoll gradient method were used for fertilization. 1 to 2×10⁶ motile sperm/ml sperm were cultured with 45 ul of an IVF-TALP medium covered with mineral oil (Nidacon, Cat. No. NO-100) in a humidifying atmosphere of 5% CO₂ with mature oocytes. Eighteen hours after in vitro fertilization, cumulus cells were removed from the zygote. This zygote was cultured in a culture medium protected by two levels of chemically defined mineral oil at a temperature of 38.5° C. in a 5% O₂, 5% CO₂, and 90% N₂ atmosphere. The zygote is cultured into an embryo.

### [Example 4] Microinjection

When the microinjection method was performed, Cas9 mRNA and sgRNA were divided into 4 groups to find the most suitable concentration. (CB; TE only microinjection, RNA1X; Cas9 mRNA: 100 ng/μL, sgRNA: 50 ng/μL, RNA2X; Cas9 mRNA: 200 ng/μL, sgRNA: 100 ng/μL, RNA 4X; Cas9 mRNA: 400 ng/μL, sgRNA: 200 ng/μL). After 18 hours of in vitro fertilization, Cas9 mRNA (sigma-Aldrich, Cat. No. CAS9MRNA) and sgRNA were synthesized by GeneArt Precision gRNA Synthesis Kit (Thermofisher, Cat. No. A29377) and injected with a microinjector machine (Eppendorf, Femtojet®) into zygotes. Seven days after microinjection, preimplantation embryos were collected and myostatin deletion was observed or implanted into the uterus of a surrogate mother.

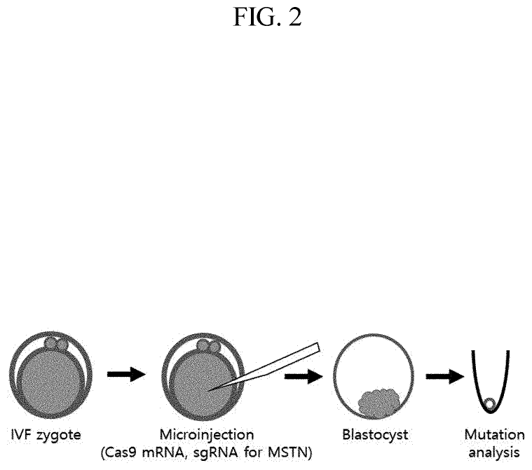

The microinjection method is illustrated in FIG. 2.

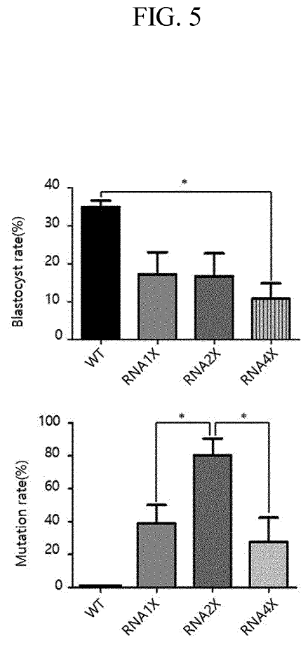

In FIG. 5, the results of the experiment by dividing Cas9 mRNA and sgRNA into four groups are schematically illustrated.

From the above results, the proportion of blastocysts was similar to that of a wild-type in both RNA1X and RNA2X. When looking at the modification ratio, the RNA2X group showed a significantly higher modification ratio than the other RNA1X and RNA4X groups. Therefore, it was determined that the concentration of RNA2X was the most suitable, and then the experiment was conducted with the concentrations of Cas9 mRNA: 200 ng/μl and sgRNA: 100 ng/μl used in the RNA2X group.

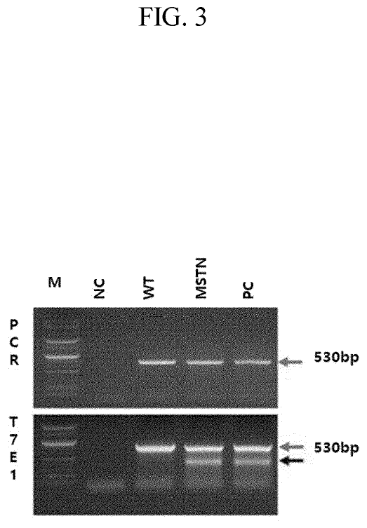

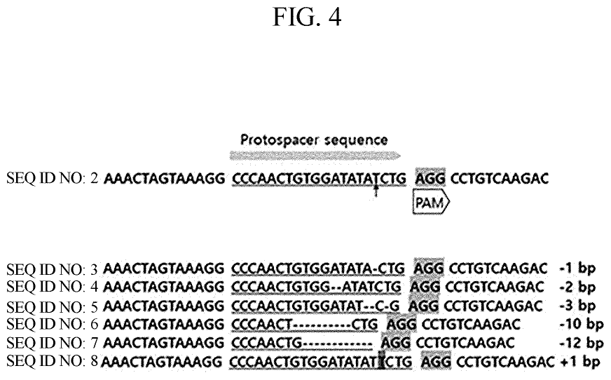

In FIGS. 3 and 4, myostatin modification of embryos after microinjection was observed.

FIG. 3 shows that when the myostatin of the embryo is modified by performing the T7E1 assay, one more band below 530 bp is observed, unlike the wild-type, the same as in the positive control. As a result of the above, it can be confirmed that the myostatin gene is modified in the embryo.

FIG. 4 is a view showing the results of myostatin-modified forms of embryos obtained by performing Sanger sequencing.

As can be seen from the above results, in embryos, 1, 2, 3, 10, and 12 base pairs of the second exon of the myostatin gene were deleted, and one base pair of a second exon of the myostatin gene was inserted.

In this way, it was confirmed that embryos, unlike animals, can have various modified forms, as well as a form in which only 12 base pairs of the second exon of the myostatin gene are deleted as a result of indels. However, the engineered cell of the present application refers only to an engineered cell having a myostatin gene in which 12 base pairs of a second exon are deleted.

### [Example 5] Embryo Transfer and Pregnancy Diagnosis

Blastocysts were stored in PBS supplemented with 20% FBS. The blastocyst was transferred to a uterus of each surrogate mother by the cervical method around day 7 (estrus=day 0=day of fusion) by a non-surgical approach. Surrogate mothers were examined by rectal palpation and ultrasound at 50 days after estrus to observe embryo survival and pregnancy. Pregnant cattles were checked periodically thereafter by rectal palpation and ultrasound.

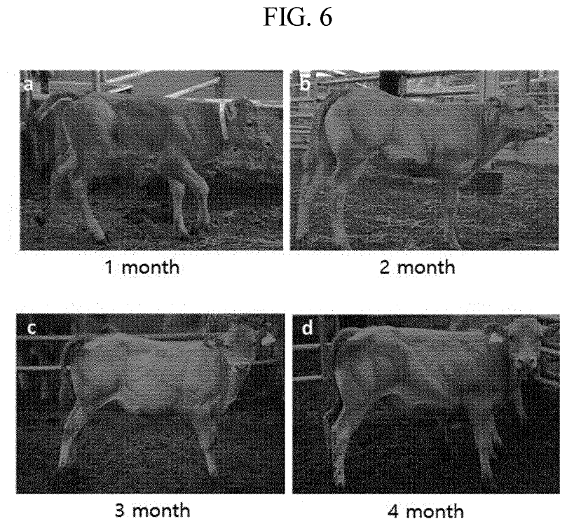

After birth, in order to confirm the change in appearance in the growth process of cattle No. 17 in FIG. 6, the change in appearance was photographed at intervals of one month from one month after birth to four months of age.

As can be seen in the above photo, cattle No. 17 is a cattle in which 12 base pairs of the second exon of the myostatin gene have been deleted, and the muscle development is visible, with a more pronounced appearance of muscle development after 3 months.

### [Example 6] T7E1 Assay

Genomic DNA obtained from transgenic primary cells was extracted using a DNA extraction kit (DNeasy Blood & Tissue kit, Qiagen, Cat. No. 69504). MSTN Primer was designed by PRIMER3 software. The PCR conditions were 94° C. for 5 minutes, 94° C. for 20 seconds, 57° C. for 30 seconds, 72° C. for 35 seconds, and 72° C. for 5 minutes for 35 to 40 cycles.

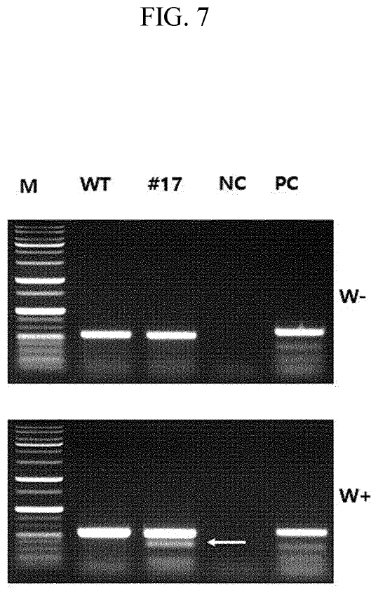

In FIG. 7, the result of 12 base pairs of myostatin gene of cattle No. 17 among the cattles born in Example 5 was confirmed by T7E1 assay.

Through the above results, it was confirmed that cattle No. 17 had a different location of the cut band as a result of the T7E1 assay, unlike the wild-type, and as a result, cattle 17 had a modified myostatin gene.

### [Example 7] Gene Expression by Real-Time PCR

Total RNA was extracted from primary cultured cells using RNeasy® Mini Kit (Qiagen, Cat. No. 74106), and complementary DNA was synthesized from 1 μg of RNA to cDNA EcoDry™ Premix (OligodT) using RNA (Takara, Cat. 639543). Gene expression analysis was performed using the SYBR Green method in QuantStudio 3 (Applied Biosystems, model number A28132), and the relative cycle threshold (CT) value was normalized by GAPDH. Primers used in the above examples are listed in SEQ ID NO: 85 to SEQ ID NO: 88.

TABLE 6

| Gene | Forward primer | Reverse primer | Size |
|---|---|---|---|
| MSTN | AACAGCGAGCAGAA | CCAGGCGAAGTTT | 124 |
| | GGAAAA | ACTGAGG | |
| | (SEQ ID NO: 85) | (SEQ ID NO: 86) | |
| GAPDH | GGCGTGAACCACGA | CCCTCCACGATGC | 120 |
| | GAAGTA | CAAAGT | |
| | (SEQ ID NO: 87) | (SEQ ID NO: 88) | |

### [Example 8] MSIN Off-Target Effect Analysis

Potential off-target effects due to CRISPR/Cas9 in three MSTN mutant calves were scanned using Cas-OFFinder software, a fast and versatile algorithm that searches for potential off-target sites of Cas9 RNA-derived endonucleases. In the MSTN target site used in the experiment, the number of mismatches was set to 3, and five base sequences were found targeting the entire gene of the cattle. Primers targeting each of the five base sequences were named SEQ ID NO: 89 to SEQ ID NO: 100, and the off-target effect according to the location of the primer was confirmed through T7E1 analysis.

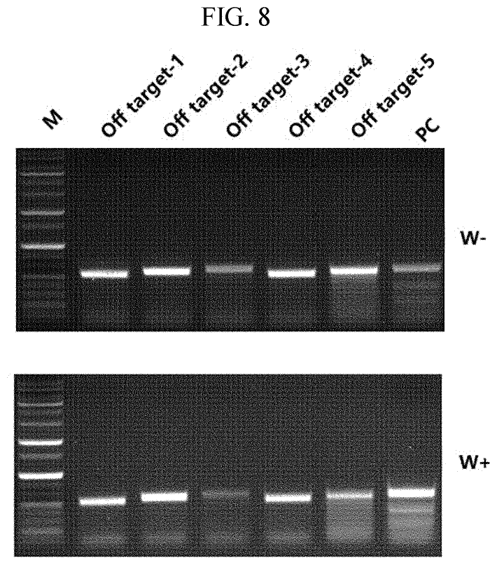

In FIG. 8, the T7E1 assay was performed as a primer sequence for the following potential off-target effect positions.

As can be seen from the results, unlike positive control, the truncated band was not identified at all five positions to confirm off-target effects. Therefore, as a result, it was confirmed that there was no off-target effect of CRISPR/Cas9 on the potential off-target position and that the guide RNA and Cas9 mRNA used in one embodiment of this application worked target-specifically.

TABLE 7

| Gene | Chromosome | Position | Forward primer | Reverse primer | Size |
|---|---|---|---|---|---|
| MSTN | 2 | 6281284 | GAGGTGTTCGTTCGTTTTT | CTACCAGTTTCCTGTGCT | 538 |
| | | | C | TA | |
| | | | (SEQ ID NO: 89) | (SEQ ID NO: 90) | |
| Off- | 2 | 6590416 | TCAGCACAGAAAAGGTGAG | GAGACGGACACAACTGAG | 515 |
| target 1 | | | G (SEQ ID NO: 91) | CA | |
| | | | | (SEQ ID NO: 92) | |
| Off- | 4 | 14356304 | TGAGCCCCTACTTTGTGGA | GTTTTCTGGTAAGGGGTG | 580 |
| target 2 | | | C (SEQ ID NO: 93) | CA | |
| | | | | (SEQ ID NO: 94) | |
| Off- | 8 | 51204411 | TTGAAAACCTAGTGGGGAA | GCACTCTCAAACACTGTG | 591 |
| target 3 | | | AAA (SEQ ID NO: 95) | GC | |
| | | | | (SEQ ID NO: 96) | |
| Off- | 9 | 88117377 | TCCTTGCACCTTCCAAAAT | ATCTGCGTGTAACTCCAG | 520 |
| target 4 | | | C (SEQ ID NO: 97) | CC | |
| | | | | (SEQ ID NO: 98) | |
| Off- | 2 | 46946387 | TCACCCATTCCAGTCCATT | CCTCTAATGCCCTCTTGC | 542 |
| target 5 | | | T (SEQ ID NO: 99) | AG | |
| | | | | (SEQ ID NO: 100) | |

### [Example 9] Targeted Deep Sequencing

According to the manufacturer's protocol, a target site was first amplified to a size of about 500 bp from an extracted genomic DNA using KAPA HiFi HotStart DNA polymerase (Roche, Cat. No. #KK2502). Ampulicon was then re-amplified to a size of up to 230 bp, and then Ampulicon was amplified using a TruSeq HT double-indexed primer to add adapters and index sequences for the Illumina sequencing platform to each sample. Primers used in this study are listed in SEQ ID NO: 101 and SEQ ID NO: 102. The pooled PCR ampoule recon was purified using the PCR Refining Kit (MGmed) and sequenced in MiniSeq (Illumina) with a paired end sequencing system (2×150 bp). Cas-Analyzer was used to quantify indel ratios in deep-sequencing data.

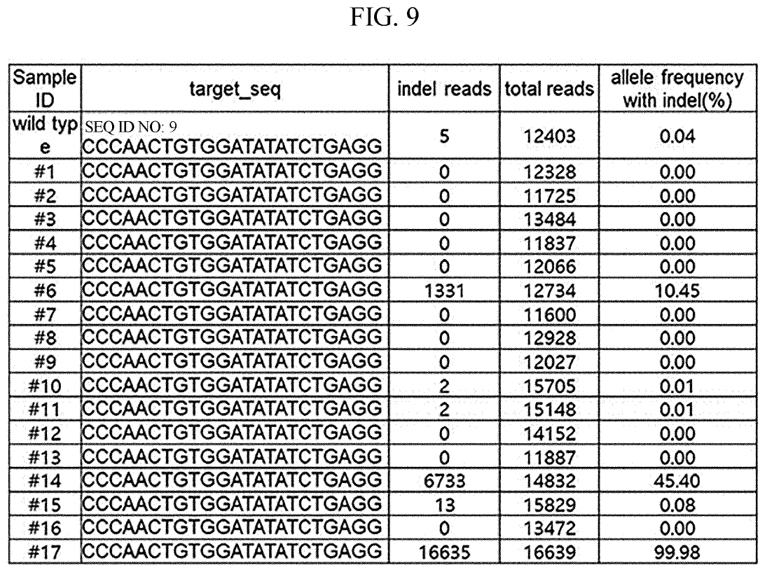

Target deep sequencing results can be confirmed in FIGS. 9 to 13.

FIG. 9 shows the results of targeted deep sequencing of 17 cattles and wild-type cattles born by transferring an engineered embryo comprising an modified myostatin gene of the present application into surrogate mothers. As can be seen from these results, unlike wild-type cattles, it can be seen that the indel ratios of cattles No. 6, No. 14, and No. 17 were 10.45%, 45.4%, and 99.98%.

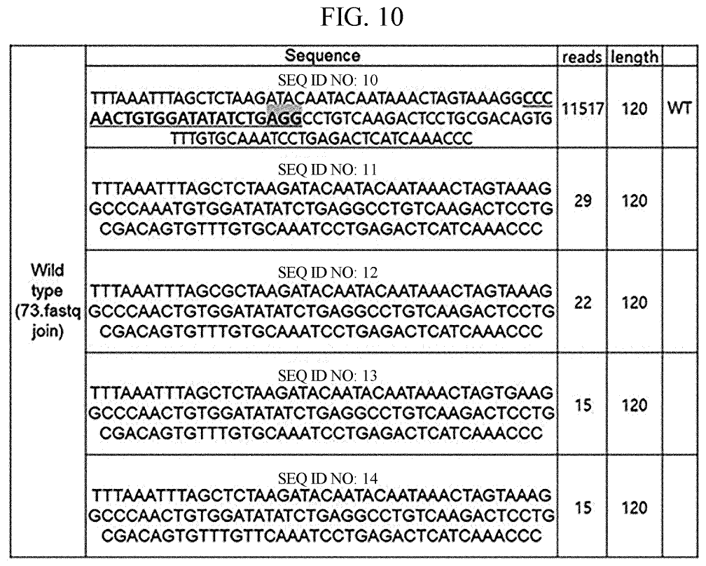

The results of listing the results of FIG. 9 in more detail can be confirmed in FIGS. 10 to 13.

Referring to FIG. 10, it was confirmed that the base pair of the second exon of the myostatin gene was not modified in wild-type cattles. Gray boxes indicate PAM sequences. The underlined sequence Gray boxes and underlines are used is the protospacer sequence. identically in FIGS. 10 to 13.

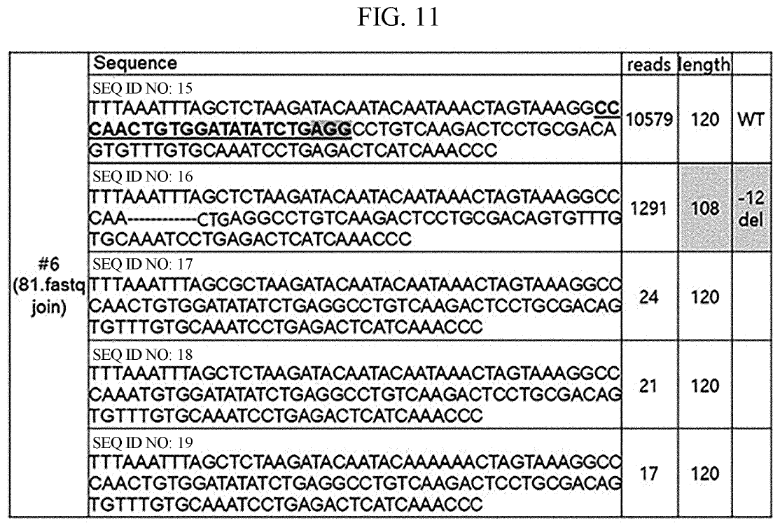

In FIG. 11, it was confirmed that 12 base pairs among the base pairs of the second exon of No. 6 cattle myostatin gene were deleted. When the ratio of indel was confirmed, 12 base pairs were deleted at 10.45%, and the read result could be confirmed.

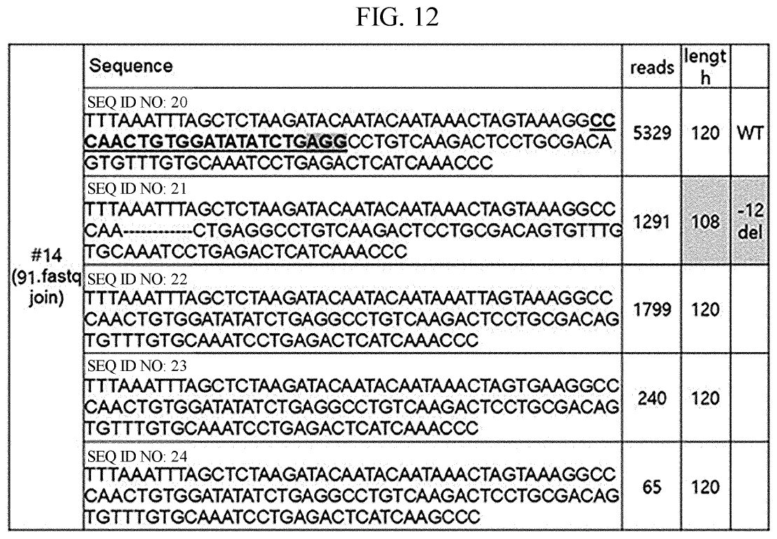

In FIG. 12, it can be seen in cattle No. 14 that 12 base pairs among the base pairs of the second exon of the myostatin gene in the same form as in FIG. 11 are determined. The indel ratio of cattle No. 14 was 45.4%.

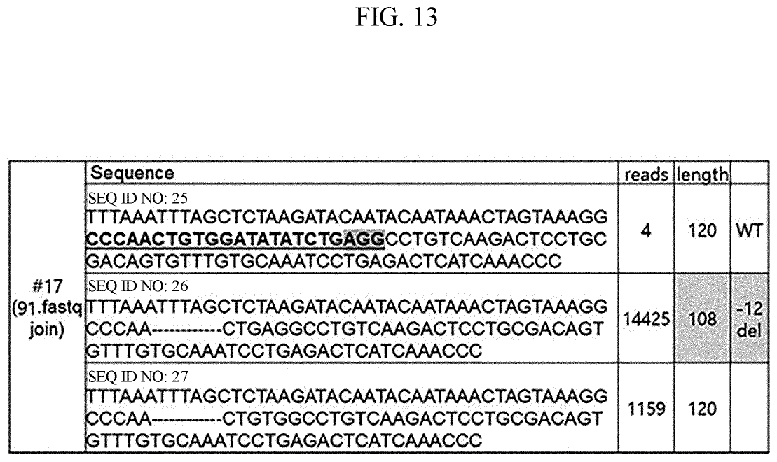

In FIG. 13, it was confirmed that 12 base pairs among the base pairs of the second exon of cattle myostatin gene number 17 were deleted. The indel ratio of cattle No. 17 was confirmed to be 99.98%. Through the above results, in this application, the target deep sequencing of the cattle born after inducing the modification of the second exon of the myostatin confirmed that the base of the second exon of the myostatin was not modified. However, it was confirmed that in all three cattles whose modification was confirmed, only 12 base pairs of the second exon were found to be deleted.

TABLE 8

| Gene | Forward primer | Reverse primer |
|---|---|---|
| MSTN-1 | GAGGTGTTCGTTCG | TAAGCACAGGAA |
| | TTTTTC (SEQ | ACTGGTAG (SEQ |
| | ID NO: 101) | ID NO: 102) |
| MSTN-2 | ACACTCTTTCCCTA | GTGACTGGAGTT |
| | CACGACGCTCTTC | CAGACGTGTGCC |
| | CGATCTaacgcaa | TTCTCGATCTtg |
| | gtggaaggaaaac | ctctgccaaata |
| | | ccagtg |
| | (SEQ ID NO: 103) | (SEQ ID NO: 104) |

### [Example 10] Primary Cell Culture

Primary cells derived from cattle's ear skin were obtained with a biopsy punch. The ear shells obtained from cattle were cut into small pieces with a sterile scalpel, washed several times, and cultured at 38° C. for 4 to 18 hours at a balanced salt solution (Gibco, Cat. No. 14175095) of HANK supplemented with collagenase (Collagenase type I, Gibco, Cat. No. 17-017). After overnight, the dispersed cells were washed several times in DMEM (Gibco, Cat. No. 21068028) medium and supplemented with 10% fetal cattle serum (Gibco, Cat. No. GIB-11150-059), 1% Penicillin/streptomycin (Gibco, Cat. No. GIB-11150-059). Cat. no. 15140148), 1% Non-essential amino acids (Gibco, Cat. No. 11140050), and 100 mM β-Mercaptoethanol (Sigma-aldrich, Cat. No. M3418).

In FIG. 7, where the T7E1 assay was performed during the process of the above example, it was performed after the primary cell culture.

In FIG. 14, after culturing the primary cells of cattles No. 14 and No. 17 born in this application, the amount of myostatin mRNA expression in wild-type cattle and cattles No. 14 and No. 17 is compared and schematized.

As can be seen from the above results, the amount of myostatin mRNA expression in primary cells of cattles No. 14 and No. 17 was reduced by more than 60% in the case of cattle No. 14 and 80% in the case of cattle NO. 17.

Therefore, it was confirmed that myostatin mRNA expression was reduced in a cattle in which 12 base pairs of the second exon of the myostatin gene of the present application were deleted compared to wild-type cattle.

### [Example 11] Germline Transmission of MSTN Knockout Cattle s

### Experiment Method

### 1) Donor Management and Egg Collection (Ovum Pick Up; OPU)

2.0 mg of estradiol benzoate was intramuscularly injected into MSTN mutant donor cattles with random estrous cycles implanted with an intravaginal progesterone device (Repro360, Cue-mate). Donor was given 200 mg of FSH (Kawasaki Pharm, Antonin R-10) divided into four doses every 12 hours (57, 57, 43 and 43 mg) on Day 4 and 5. A P4 device was immediately removed on day 7 prior to OPU.

For the OPU the donor cattle was restrained from the cattle crush. Epidural anesthesia was performed with 5 ml, 2% lidocaine (Daihan, DAIHAN Lidocaine, South Korea). The ovary was fixed by transrectal manipulation and stayed on the probe of an ultrasound device. One trained OPU technician performed the OPU procedure using an ultrasound device (Esaote, MyLab One) coupled with a 7.5 MHZ transrectal transducer probe with a follicular aspiration guide (WTA, catalog number 10283). Follicular puncture was performed using an 18G OPU tread needle (WTA, catalog number 17927) and follicular fluid was collected in a 50 ml tube. Oocytes from the follicular fluid were collected under a stereomicroscope and used for in vitro fertilization. The remaining hair follicle fragments were used for primary culture.

### 2) Semen Collection

Semen was collected from MSTN mutant bulls using electroejaculation. (3 times per bull). Before semen collection, the foreskin was cut, the orifice was washed with clean water, and then dried with a clean paper towel to minimize contamination. Electro ejaculation was performed using a manually controlled electro ejaculator ElectroJac6 (Ideal® Instruments Neogen Corporation, Lansing, MI, USA) with an attached 6.5 cm diameter rectal probe, three ventral electrodes spaced approximately 1 cm apart, and electrodes were fully inserted into the rectum facing the abdomen. The number of electrical stimuli was increased until the bull ejaculated. Each stimulus lasted 8 to 10 seconds and was paused for approximately 2.0 seconds before the next stimulus was applied. When the semen secretion became cloudy, the collection tube was placed over the penis to collect the semen. Ejaculated semen was transported to the laboratory at 25° C. within 30 minutes.

### 3) Semen Cryopreservation and Thawing

Semen samples were used for cryopreservation when they showed a general motility of 60% or more. Semen samples were expanded with Optixcell® (IMV Technologies) at 37° C. The expanded semen was equilibrated at 4° C. for 3 hours and then placed in a 0.5 ml straw. The filled straws were placed on a special rack at a height of 5 cm above liquid nitrogen, exposed to liquid nitrogen vapor for 15 minutes, and then placed in a cryogenic tank filled with liquid nitrogen (−196° C.). Cryopreserved sperm were thawed in a water bath at 37° C. for 45 seconds.

### 4) Sperm Motility Assay

To analyze and quantify sperm motility, the IVOS-II CASA (Computer Assisted Sperm Analysis Program) system was used according to the manufacturer's instructions. Briefly, frozen semen was thawed, incubated, and purified using the same protocol used for IVF. Then, 3 μl of sperm was loaded into a sperm analysis chamber (Leja slide) and analyzed by CASA. Frozen straws from three different bulls were used. To rule out technical errors, each semen was analyzed three times, and an average value of the CASA results was used for statistical evaluation.

### 5) In Vitro Fertilization and Culture

Motile sperm were selected using the Percoll gradient method as previously described. Briefly, frozen thawed semen from F0 bulls at 35° C. was filtered by centrifugation at 1680 rpm for 15 min on a Percoll discontinuous gradient (45% to 90%). To produce a 45% Percoll solution, 1 mL of capacitation-Tyrode's albumin lactate pyruvate (TALP) medium was added to 1 mL of 90% Percoll. Sperm pellets were washed twice with 3 mL of volumetric-TALP medium and centrifuged at 1680 rpm for 5 minutes. Washed motile sperm were used for IVF. Sperm (1 to 2×10⁶ sperm/mL) were cultured with mature oocytes for 18 hours in 50 μL IVF-TALP medium covered with mineral oil (Nidacon, catalog number NO-100) in a 5% humid atmosphere. Cumulus cells were removed from putative zygotes after 18 hours of co-incubation at 38.5° C. CO₂. Zygotes were cultured in a two-stage chemically defined culture medium covered with mineral oil in an atmosphere of 5% O₂, 5% CO₂, and 90% N₂ at 38.5° C.

### Experiment Result

### 1) Germline Transmission of MSTN Mutant Female Cattle

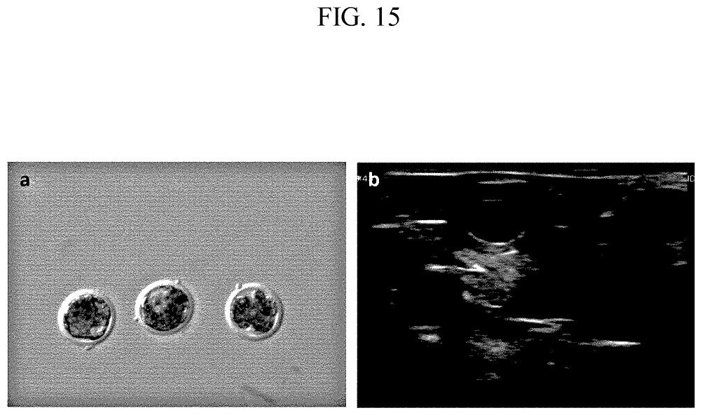

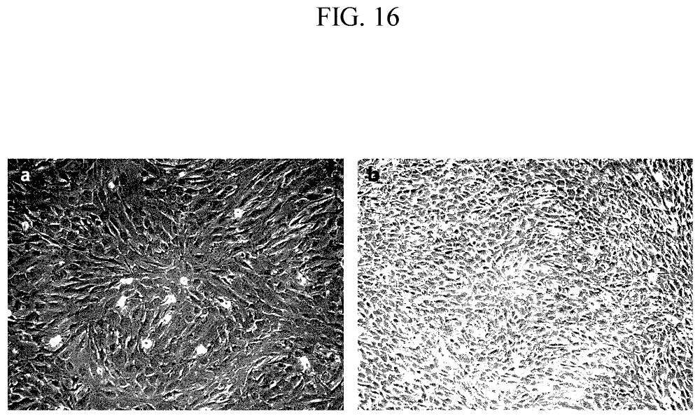

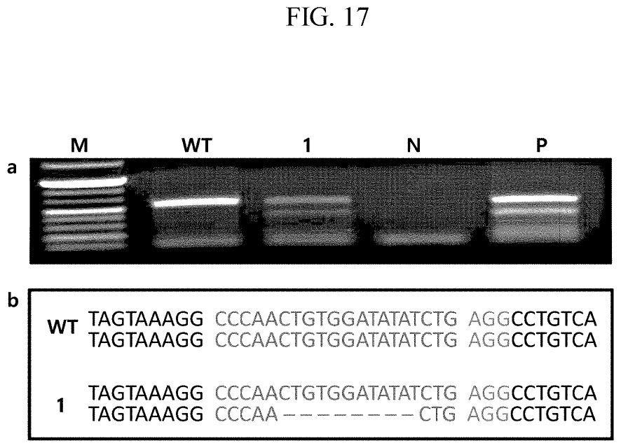

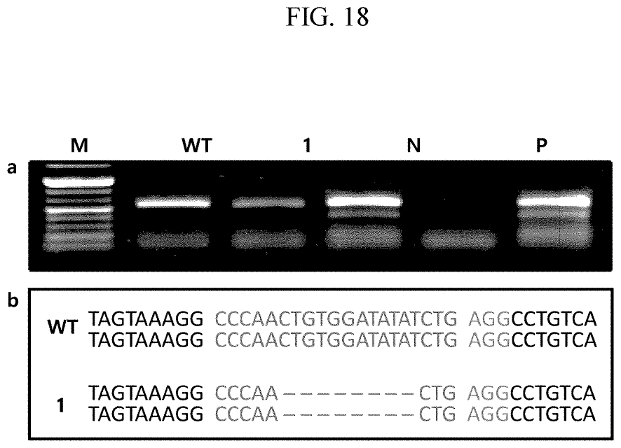

A total of 45 oocytes were collected after OPU was performed (n=3). After in vitro fertilization with wild-type freeze-thawed semen, 45 oocytes were cultured, and 5 blastocysts (12.5±10.9%) were formed (FIG. 15A). Selected blastocysts were transferred to 5 recipients. Pregnancy was confirmed in one recipient by rectal palpation and ultrasound (FIG. 15B). In addition, MSTN mutations were confirmed by culturing follicular fluid obtained during OPU (FIG. 16). T7E1 assay and Sanger sequencing confirmed the same mutations as in F0 females in both remaining embryos and follicular fluid-derived cells (FIGS. 17 and 18). These results demonstrate successful germline transmission in MSTN mutant cattle using the OPU procedure.

FIG. 15 shows the results of germline transmission of MSTN mutant females, showing the production of MSTN mutant blastocysts derived from MSTN mutant cattle oocytes (A) and representative photographs of pregnancy diagnosis using an ultrasound machine on day 30 (B).

FIG. 16 is a somatic cell image derived from follicular fluid obtained by the OPU process ((a): MSTN mutant female, (b): wild-type).

FIG. 17 shows the T7E1 assay results (a) and sequencing data (b) of MSTN mutant female blastocysts (M: marker; WT: wild-type; 1: MSTN mutant female; N: negative control group; P: T7E1 positive control group).

FIG. 18 shows the results of the T7E1 assay (a) and sequencing data from somatic cells of follicular fluid (b) (1: MSTN mutant female (no wild-type); 2: MSTN mutant female (no wild-type)).

### 2) Germline Transmission of MSTN Mutant Male

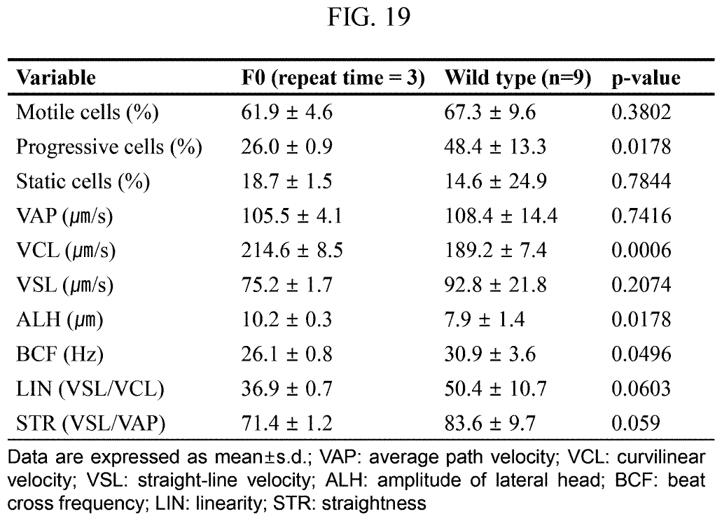

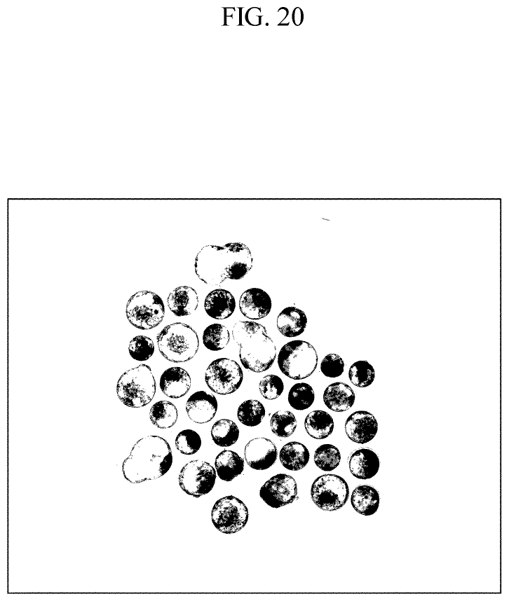

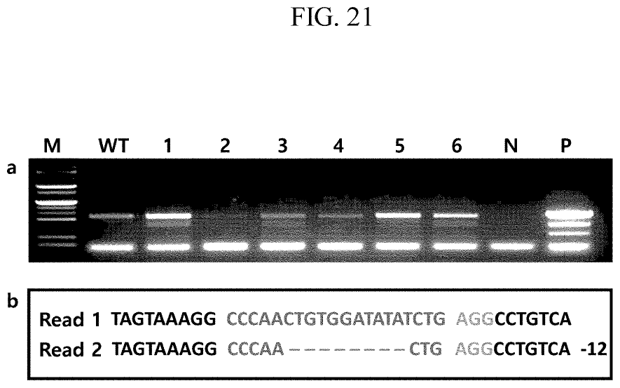

Semen was collected by electroejaculation from male bulls (F0) with a 10.5% mutation. Samples were frozen for in vitro fertilization and thawed for sperm motility. CASA showed significant differences in advanced cells (%), VCL, ALH, and BCF between F0 and wild-type samples. However, LIN and STR did not show significant differences between F0 and wild-type samples (FIG. 19). Furthermore, no detrimental effects on embryo development and competence have been shown when semen samples are used for in vitro fertilization. Oocytes collected from slaughterhouses were fertilized with frozen-thawed semen and cultured to develop into blastocysts. A total of 335 oocytes (number of replicates=3) were used. Among them, 261 oocytes (78.9±10.8%) were cleaved, and 166 blastocysts (50.5±6.8%) were formed (FIG. 20). Total cell number was 81.3±20.6 (n=20). Mutations were analyzed in 117 blastocysts, and 15 blastocysts showed MSTN mutations (12.7±3.1%) (FIG. 21).

FIG. 19 shows a summary of semen from the MSTN male founder by Computer Assisted Semen Analysis.

FIG. 20 shows a photograph of a representative blastocyst (blastocyst produced in vitro fertilized with MSTN mutant bull semen) as a validated result of germline transfer from an MSTN mutant male cattle.

FIG. 21 shows the mutation rate of the MSTN gene in blastocysts derived from in vitro fertilized MSTN mutant bull semen (1 to 6: randomly selected blastocysts). The top panel (a) shows the T7E1 results, and the bottom panel (b) shows the sequencing results of the MSTN target site.

1-34. (canceled)

35. A transgenic animal comprising an artificially modified myostatin gene,
wherein the artificial modification is located in a second exon of the myostatin gene,
wherein the modification is a deletion of 12 base pairs corresponding to a region encoding an amino acid sequence of the order of leucine, tryptophan, isoleucine and tyrosine (LWIY) in the second exons, compared to an amino acid sequence of a wild-type pro-myostatin protein,
wherein a nucleic acid sequence of the artificially modified myostatin gene encodes an engineered pro-myostatin protein consisting of amino acid sequence selected from SEQ ID NOs: 30 to 33,
wherein the transgenic animal expresses the engineered pro-myostatin protein.

36. The transgenic animal of claim 35,
wherein an amount of myostatin mRNA expression in the transgenic animal is less than that of a wild-type animal.

37. The transgenic animal of claim 35,
wherein amino acid sequence of the engineered pro-myostatin protein lacks an amino acid sequence of LWIY compared to amino acid sequence of wild-type pro-myostatin protein.

38. The transgenic animal of claim 35,
wherein muscle mass of the transgenic animal is higher than a wild-type animal.

39. An engineered cell comprising a nucleic acid encoding an engineered pro-myostatin protein consisting of amino acid sequence selected from SEQ ID NOs: 30 to 33,
wherein amino acid sequence of the engineered pro-myostatin protein lacks an amino acid sequence of LWIY compared to amino acid sequence of wild-type pro-myostatin protein.

40. An engineered pro-myostatin protein in which consequence four amino acids in the sequence of leucine, tryptophan, isoleucine, and tyrosine are deleted compared to wild-type pro-myostatin protein,
wherein an amino acid sequence of the engineered pro-myostatin protein is a sequence of SEQ ID NOs: 30 to 33.

41. A nucleic acid encoding the engineered pro-myostatin protein of claim 40.
